(19) United States
(12) Patent Application Publication
(10) Pub. No.: US 2023/0165269 A1
(43) Pub. Date: Jun. 1, 2023

(54) CHEWABLE MATRIX AND USE THEREOF FOR THE COLLECTION OF SALIVA SAMPLES AND INFECTION DETECTION

(71) Applicant: WM. WRIGLEY JR. COMPANY, Chicago, IL (US)

(72) Inventors: David R. PHILLIPS, Chicago, IL (US); Jingping LIU, Chicago, IL (US); Michael W. DODDS, Chicago, IL (US)

(73) Assignee: WM. WRIGLEY JR. COMPANY, Chicago, IL (US)

(21) Appl. No.: 17/922,756

(22) PCT Filed: Apr. 28, 2021
(86) PCT No.: PCT/US2021/029529

§ 371 Date: Nov. 1, 2022

Related U.S. Application Data
(60) Provisional application No. 63/019,835, filed on May 4, 2020.

(51) Int. Cl. A23G 4/08; C12Q 1/06; A23G 4/10; A23G 4/12; C12Q 1/6883; C12Q 1/24

(52) U.S. Cl. CPC A23G 4/08; C12Q 1/06; A23G 4/10; A23G 4/123; C12Q 1/6883; C12Q 1/24

(57) ABSTRACT

The present disclosure is directed to a chewable matrix that can be used as a tool for collection of saliva samples. Also disclosed herein are methods for extracting saliva, and in particular microorganisms or nucleic acid, from the chewable matrix, methods for detecting an illness or infection in subjects using the chewable matrix, and kits for performing these methods.

### BACKGROUND OF THE DISCLOSURE

The present disclosure is directed to a chewable matrix that can be used as a tool for collection of saliva samples. Also disclosed herein are methods for extracting saliva, and in particular microorganisms or nucleic acid, from the chewable matrix, and methods for detecting an illness or infection in subjects using the chewable matrix, and kits for performing these methods.

Most current tests for determining infection with respiratory illnesses require throat, nasal, nasopharyngeal, or oropharyngeal swabs. In order to obtain swab samples, trained medical professionals need to be in direct contact with patients in either medical facilities or drive-through test sites. The total number of collectable swab samples is therefore limited by individual operations. In addition to time constraint from in-person swabbing, consumption of personal protective equipment (PPE) to protect medical professionals and consumption of sterilized cotton swabs may impose additional challenges to the medical system. As a result, the ability to scale up population screening for infection or illness and sample collection is limited. It would thus be desirable to develop a method for screening subjects for infection with respiratory or other illnesses that can be readily scaled up to rapidly test a population of subjects, and wherein the presence of a medical professional is not required for sample collection.

The human oral cavity contains a varied and vast amount of flora, and many diseases of the gastrointestinal system and respiratory system can be revealed in the oral cavity. There are also many diseases that are specific to the oral cavity. In addition to bacterial organisms, oral microorganisms can include fungal, protozoal, and viral species and many of these microorganisms adhere to the teeth, the gingival sulcus, the tongue, and the buccal mucosa. Each site has a unique way of allowing the organisms to establish their residency.

Certain microorganisms, such as viruses, may be present in epithelial cells in the oral cavity. The main host cell receptor for severe acute respiratory syndrome coronavirus 2 (SARS-CoV-2), which causes COVID-19 respiratory disease, is the ACE2 receptor. High expression levels of the ACE2 receptor have been found on the epithelial cells of oral mucosa, and especially the tongue. SARS-CoV-2 has also been found floating free in saliva. It is also known that the presence of bacterial cells and epithelial cells in saliva increases upon gum chewing. See, e.g., Dawes et al., Archives of Oral Biology, 2001, Vol. 46, pp. 625-632. Since epithelial cells from the oral cavity may be found in saliva, increasing the production of saliva or increasing the presence of epithelial cells or microorganisms in saliva would be an improvement over traditional methods of saliva collection, and may provide increased sensitivity to diagnostic tests performed on saliva for the purpose of diagnosing an illness.

Many microorganisms may adhere to polymers that they contact while in the oral cavity. It is also known that conventional chewable polymer-based confectionery products can trap saliva within the polymer. Such products, however, commonly contain certain components that may interfere with its use as a tool for collecting saliva samples. For instance, certain flavors present in chewable polymer-based confectionery products can reduce the activity of microorganisms, such as a virus, thus making any saliva sample collected less effective for use in diagnosing infections or illness. Additionally, such confectionery products typically contain a large amount of bulk sweetener. The presence of large amounts of bulk sweeteners may reduce the content of microorganisms that initially interact with the polymer due to the sweetener being dissolved and swallowed in the initial stages of chewing.

There is thus a need for the development of new polymers that can be used for collection of saliva samples, and for methods of detecting, identifying, and quantifying the numbers and types of microorganisms that adhere to or are entrapped within such polymers in the oral cavity. There is a further need for methods for increasing the production of saliva in a subject and for increasing the presence of epithelial cells and/or microorganisms in a saliva sample to assist with infection detection methods. There is also a need for an easy to use, scalable, shelf-stable method for collection of saliva samples from a population of subjects.

### SUMMARY

The present disclosure is directed to a chewable matrix that can be used as a tool for collection of saliva samples. Also disclosed herein are methods for extracting saliva, and in particular microorganisms or nucleic acid, from the chewable matrix, and methods for detecting an illness or infection in subjects using the chewable matrix, and kits for performing these methods.

In one aspect, the present disclosure is directed to a chewable matrix comprising low molecular weight polyvinyl acetate in an amount of from about 5% to about 40% by weight of the matrix, and de-oiled lecithin in an amount of from about 13% to about 50% by weight of the matrix.

In another aspect, the present disclosure is directed to a chewable matrix comprising low molecular weight polyvinyl acetate and a low molecular weight elastomer.

In another aspect, the present disclosure is directed to a chewable matrix comprising low molecular weight polyvinyl acetate and at least one plasticizer, wherein the matrix is essentially free of elastomers.

In another aspect, the present disclosure is directed to a method of collecting a saliva sample from a subject, the method comprising contacting the oral cavity of the subject with a chewable matrix of the present disclosure.

In another aspect, the present disclosure is directed to a method of detecting an illness or infection in a subject, the method comprising contacting the oral cavity of the subject with a chewable matrix of the present disclosure.

In an aspect, which may be used in combination with each or any of the above-mentioned aspects, microorganisms are extracted from the chewable matrix by sonicating the chewable matrix under conditions that remove the microorganisms from the chewable matrix and deposit the microorganisms in a suspension.

In an aspect, which may be used in combination with each or any of the above-mentioned aspects, at least a portion of the suspension is contacted with a solid support under conditions that promote colony formation.

In an aspect, which may be used in combination with each or any of the above-mentioned aspects, microorganisms are extracted from the chewable matrix by contacting the chewable matrix with a solution comprising at least one selected from the group consisting of an organic solvent, a buffer, and water.

In an aspect, which may be used in combination with each or any of the above-mentioned aspects, nucleic acids are extracted from the chewable matrix by contacting the chewable matrix with a solution comprising at least one selected from the group consisting of an organic solvent, a buffer, and water, and optionally subjecting to a mechanical mixer to break up the chewable matrix.

In an aspect, which may be used in combination with each or any of the above-mentioned aspects, the organic solvent is chloroform.

In an aspect, which may be used in combination with each or any of the above-mentioned aspects, the source of the nucleic acids extracted from the chewable matrix is identified using polymerase chain reaction.

In an aspect, which may be used in combination with each or any of the above-mentioned aspects, the identified source of the nucleic acids is a virus.

In an aspect, which may be used in combination with each or any of the above-mentioned aspects, the virus is severe acute respiratory syndrome coronavirus 2 (SARS-CoV-2).

In an aspect, which may be used in combination with each or any of the above-mentioned aspects, the nucleic acids extracted from the chewable matrix is quantified using quantitative polymerase chain reaction.

In an aspect, which may be used in combination with each or any of the above-mentioned aspects, microorganisms adhered to or entrapped within the chewable matrix following mastication are detected by (a) dissolving at least a portion of the chewable matrix in a solution comprising at least one selected from the group consisting of an organic solvent, a buffer, and water, (b) extracting nucleic acids from the microorganisms, and (c) amplifying the nucleic acids using at least one oligonucleotide primer specific for the microorganism.

In an aspect, which may be used in combination with each or any of the above-mentioned aspects, the nucleic acids are amplified using polymerase chain reaction.

In an aspect, which may be used in combination with each or any of the above-mentioned aspects, microorganisms adhered to or entrapped within the chewable matrix are quantitated by (a) sonicating the chewable matrix under conditions that remove the microorganisms from the chewable matrix and deposit the microorganisms in a suspension, (b) contacting at least a portion of the suspension with a solid-support under conditions that promote colony formation, and (c) quantitating the colonies formed on the solid-support, wherein the number of colonies indicates the total quantity of microorganisms.

In an aspect, which may be used in combination with each or any of the above-mentioned aspects, the microorganisms are selected from the group consisting of bacteria, virus, fungus, protozoa, and combinations thereof.

In another aspect, the present disclosure is directed to a kit for detection of illness or infection, the kit comprising a chewable matrix of the present disclosure; at least one container for collection of a saliva sample following mastication of the chewable matrix; and components for detection and/or quantification of microorganisms and/or nucleic acids.

### DETAILED DESCRIPTION OF THE DISCLOSURE

In one aspect, the present disclosure is directed to a chewable matrix that can be used as a tool for the collection of saliva samples from a subject. The chewable matrix of the present disclosure is flexible and pliable at the temperature and pH within a living organism but is stable when exposed to varying forces and conditions (e.g. temperature, pH, enzymes) applied by the organism. For example, the chewable matrix is malleable in response to force applied by the organism (e.g. chewing, grinding or gnashing of the teeth or gums), the temperature in the organism (e.g. the temperature of the oral cavity of the organism) or chemical conditions (e.g. enzymes produced by the organism, pH of the oral cavity of the living organism).

The chewable matrix of the present disclosure advantageously can be used as a tool for the diagnosis of illness or infection and for the collection of saliva samples from a subject. As used herein, the term “subject” refers to a living organism, such as a mammal, and more particularly a human. For instance, the human oral cavity contains a varied and vast amount of flora, and many diseases of the gastrointestinal system and respiratory system can be revealed in the oral cavity. Many of these microorganisms may adhere to polymers that they contact while in the oral cavity. Additionally, mastication of chewable polymers can increase the production of saliva in the oral cavity, as well as the levels of epithelial cells and microorganisms in saliva. The chewable matrix of the present disclosure can thus be used as an alternative to traditional saliva collection or oral cell swabbing techniques.

The chewable matrix of the present disclosure can assist in saliva sample collection in two different ways. In one embodiment, upon contact with an oral cavity, and specifically upon mastication, saliva and microorganisms present in the oral cavity become adhered to or entrapped within the chewable matrix. The phrase “adhered to or entrapped within” a chewable matrix includes instances where the microorganisms and/or saliva are attached/adhered to the outer surface of the chewable matrix and/or are attached/adhered to and/or are embedded/entrapped within an internal surface of the chewable matrix or within folds of the chewable matrix. The saliva and microorganisms can then be extracted from the chewable matrix, and the microorganisms identified and/or quantified using techniques, such as those described herein. Advantageously, because the tongue remains in direct contact with the chewable matrix during mastication, exposure to microorganisms present in the oral cavity is maximized.

In another embodiment, the chewable matrix can be used to hydrate the oral cavity and stimulate the production of saliva in a subject. In particular, it has been discovered that the chewable matrix of the present disclosure hydrates the oral cavity by increasing the production of saliva during mastication. Because the quantity of saliva in the oral cavity is higher following mastication of the chewable matrix, collection of saliva samples is facilitated. For instance, certain microorganisms, such as viruses, may be present in epithelial cells in the oral cavity. Since epithelial cells from the oral cavity may be found in saliva, increasing the production of saliva increases the presence of epithelial cells in the saliva sample. Mastication of the chewable matrix may also act to dislodge epithelial cells or other microorganisms (e.g., bacteria, viruses, etc.) from the oral mucosa, thus increasing their concentration in saliva. The increased saliva output and increased amount of epithelial cells and/or microorganisms in saliva that is realized following mastication of the chewable matrix of the present disclosure is an improvement over traditional saliva collection techniques, and may increase the sensitivity of detection methods used to analyze the saliva samples for illness or infection. Additionally, since numerous microorganisms can be found in the oral cavity, the chewable matrix of the present disclosure can be used to screen for multiple types of illnesses.

The chewable matrix of the present disclosure also has low maintenance and a long shelf-life and is suitable for mass home deployment and easy home use. For instance, collection of saliva samples using the chewable matrix of the present disclosure advantageously does not require the presence of a medical professional (i.e., may be done in the absence of a medical professional). As used herein, “medical professional” refers to any individual who has had formal or informal medical training, and includes nurses, doctors, emergency medical technicians, medical researchers, lab technicians, and the like. Following mastication of the chewable matrix, a subject (or other non-medical professional) may collect a saliva sample by i) providing or expectorating saliva into a container or other collection device traditionally used for the collection of saliva samples; ii) placing the masticated chewable matrix into a container or other collection device; or iii) providing or expectorating saliva into a container or other collection device and also placing the masticated chewable matrix into the same or a different container or other collection device. In embodiments where collection of the saliva sample involves placing the masticated chewable matrix into a container or other collection device, the saliva collection method may further comprise extracting saliva and/or microorganisms from the chewable matrix, using a method as set forth herein. The saliva samples collected by the methods described herein may be analyzed for the presence of microorganisms, and for diagnosing illness or infection using any technique known in the art. Advantageously, diagnostic tests performed on saliva samples collected using the chewable matrix of the present disclosure may have greater sensitivity, as compared to tests performed on saliva samples collected in a traditional manner, since use of the chewable matrix may increase the presence of epithelial cells and/or microorganisms in the saliva sample.

Thanks to low maintenance, long shelf-life, and ease of use, the chewable matrix of the present disclosure may be more amenable to scaled up testing or saliva collection efforts than traditional saliva collection or oral cell swabbing techniques, while also reducing the burden on medical professionals to be present during sample collection.

Thus, in one aspect, the present disclosure is directed to a method of collecting a saliva sample from a subject. In another aspect, the present disclosure is directed to a method of detecting an illness or infection in a subject. A non-limiting embodiment of a method of collecting a saliva sample from a subject or detecting an illness or infection in a subject comprises contacting the oral cavity of the subject with a chewable matrix of the present disclosure. In one embodiment, the method comprises masticating the chewable matrix by the subject. The chewable matrix can be masticated for any amount of time. In one embodiment, the chewable matrix is masticated for at least 2 minutes, at least 3 minutes, at least 5 minutes, at least 10 minutes, or about 3 minutes to about 10 minutes. Following mastication, the saliva sample is collected. In one embodiment, the saliva sample is collected by having the subject provide or expectorate saliva into a container or other collection device traditionally used for the collection of saliva samples following mastication of the chewable matrix. In another embodiment, the saliva sample is collected by having the subject place the masticated chewable matrix into a container or other collection device. In such an embodiment, the saliva sample is adhered to or entrapped within the masticated chewable matrix and may be extracted using any of the methods as disclosed herein. In another embodiment, the saliva sample is collected by having the subject provide or expectorate saliva into a container or other collection device following mastication of the chewable matrix and also placing the chewable matrix into the same or a different container or collection device. In one particular embodiment, the saliva sample is collected by the subject in the absence of a medical professional.

In one embodiment, the method further comprises extracting saliva and/or microorganisms and/or nucleic acids from the masticated chewable matrix. The saliva and/or microorganisms and/or nucleic acids may be extracted from the masticated chewable matrix using a method as disclosed herein. A “masticated” chewable matrix refers to a chewable matrix that has been orally chewed by a subject in vivo. In one embodiment, a saliva sample collected by the methods disclosed herein may be further analyzed for the presence of microorganisms and used for the diagnosis of illness or infection in the subject using techniques known in the art.

The methods of the present disclosure can be used to detect or identify infection with any type of microorganism including bacteria, virus, fungus and protozoa, present in the oral cavity of a subject, including those set forth hereinafter.

For example, the disclosure provides for methods of detecting pathogenic or commensal bacteria present in the oral cavity such as Streptococcus mutans, Streptococcus sanguinis, Streptococcus salivarius, Porphyromomas gingivalis, Porphymomas intermedia, Actinomyces naeslundii, Bacteroides forsythus, Tannerella forsythia, Campylobacter rectus, Eubacerium nodatum, Peptostreptococcus micros, Streptococcus intermedius, Aggregetibacter actinomycetemcomitans, Treponema denticola, Eikenella corrodens, Capnocytophaga gingivalis, Streptococcus gordonii, Veillonella parvula, Streptococcus oralis, Fusobacterium nucleatum, Scardovia wiggsiae, Prevotella intermedia, Lactobacillus salivarius, Streptococcus salivarius and Streptococcus sobrinus. Optionally, the method may further comprise the step of diagnosing a bacterial infection in a mammalian subject, such as a human subject, by detecting the presence of the bacteria in a saliva sample collected following mastication of the chewable matrix of the present disclosure, and more particularly, by detecting the presence of bacteria adhered to or entrapped within a chewable matrix that was contacted with the oral cavity (e.g., masticated) of the mammalian subject. The methods also may optionally comprise the step of informing the subject of the presence of the bacteria or the diagnosis of a bacterial infection in the oral cavity.

The disclosure also provides for an in vitro method of diagnosing a bacterial infection in the oral cavity of a mammalian subject comprising detecting a bacteria in a saliva sample collected following mastication of the chewable matrix of the present disclosure, and more particularly, by detecting a bacteria adhered to or entrapped within a chewable matrix according to a method of the present disclosure, wherein the chewable matrix was in contact with the oral cavity of subject (e.g., a masticated chewable matrix), and wherein the presence of the bacteria in the saliva sample, or more particularly, adhered to or entrapped within the chewable matrix, is indicative of a bacterial infection in the oral cavity of the subject.

The disclosure also provides for an in vitro method of determining the susceptibility of a mammal subject for developing a bacterial infection in the oral cavity comprising detecting a bacteria in a saliva sample collected following mastication of the chewable matrix of the present disclosure, and more particularly, detecting a bacteria adhered to or entrapped within a chewable matrix according to a method of the present disclosure, wherein the chewable matrix was in contact with the oral cavity of the subject (e.g., a masticated chewable matrix), and wherein the presence of the bacteria in the saliva sample, or more particularly adhered to or entrapped within the chewable matrix, is indicative of increased susceptibility for developing a bacterial infection in the oral cavity of the subject or increased susceptibility of developing a disease, condition or disorder associated with the presence of the bacteria in the oral cavity of the subject.

In addition, the disclosure provides for a method of detecting virus present in the oral cavity of a subject, wherein the virus may be Human Herpes Virus (HHV)-1 (also known as herpes simplex virus (HSV)-1), HHV-2 (HSV-2), HHV-3 (also known as Varicella-zoster virus), HHV-4 (Epstein-Barr virus), HHV-5 (cytomegalovirus), HHV-6, HHV-7, HHV-8, poliovirus, group A coxsackievirus, group B coxsackievirus, echovirus and Enterovirus 71 (EV-71), Human Papillomavirus family (HPV), e.g. HPV-16, HPV-18, HPV-33, HPV-35, mumps virus, Newcastle disease virus, human parainfluenza virus type 2, 4a and 4, Paramyxovirus, Rubivirus, human influenza virus A, e.g. H1N1 and H3N3, human influenza virus B, human influenza virus C, rhinovirus, coronaviruses, which include severe acute respiratory syndrome coronavirus (SARS-CoV), and severe acute respiratory syndrome coronavirus 2 (SARS-CoV-2, which causes COVID-19 respiratory disease), canine oral Papilloma virus, feline calicivirus, and/or feline herpesvirus. Optionally, the method may further comprise the step of diagnosing a viral infection in a subject, such as a mammal, or more particularly a human subject, by detecting the presence of the virus in a saliva sample collected following mastication of the chewable matrix of the present disclosure, and more particularly detecting the presence of the virus adhered to or entrapped within a chewable matrix that was contacted with the oral cavity of the mammal (e.g., a masticated chewable matrix). The methods also may optionally comprise the step of informing the human subject of the presence of the virus or the diagnosis of a viral infection in the oral cavity.

The disclosure also provides for an in vitro method of diagnosing a viral infection in the oral cavity of a mammalian subject comprising detecting a virus in a saliva sample collected following mastication of the chewable matrix of the present disclosure, and more particularly detecting a virus adhered to or entrapped within a chewable matrix according to a method of the present disclosure, wherein the chewable matrix was in contact with the oral cavity of subject (e.g., a masticated chewable matrix), and wherein the presence of the virus in the saliva sample, and more particularly the presence of the virus adhered to or entrapped in the chewable matrix is indicative of a viral infection in the oral cavity of the subject.

The disclosure also provides for an in vitro method of determining the susceptibility of a mammal subject for developing a viral infection in the oral cavity comprising detecting a virus in a saliva sample collected following mastication of the chewable matrix of the present disclosure, and more particularly, detecting a virus adhered to or entrapped within a chewable matrix according to a method of the present disclosure, wherein the chewable matrix was in contact with the oral cavity of the subject (e.g., a masticated chewable matrix), and wherein the presence of the virus in the saliva sample, and more particularly the presence of the virus adhered to or entrapped within the chewable matrix, is indicative of increased susceptibility for developing a viral infection in the oral cavity of the subject or increased susceptibility of developing a disease, condition or disorder associated with the presence of the virus in the oral cavity of the subject.

Further, the disclosure provides for methods of detecting the presence of a fungus in the oral cavity. The methods of the disclosure may detect a fungus such as Candida e.g. Candida albicans, Aspergillus, Cryptococcus neoformans, Cryptococcus gattii, Histoplasma capsulatum, Blastomyces dermatitidis, Paracoccidioides brasiliensis, Coccidiodes immites and Zygomycota. Optionally, the method may further comprise the step of diagnosing a fungal infection in a mammal, such as a human subject, by detecting the presence of the fungus in a saliva sample collected following mastication of the chewable matrix of the present disclosure, and more particularly detecting the presence of the fungus adhered to or entrapped within a chewable matrix that was contacted with the oral cavity of the subject (e.g., a masticated chewable matrix). The methods also may optionally comprise the step of informing a human subject of the presence of the fungus or the diagnosis of a fungal infection in the oral cavity.

The disclosure also provides for an in vitro method of diagnosing a fungal infection in the oral cavity of a mammalian subject comprising detecting a fungus in a saliva sample collected following mastication of the chewable matrix of the present disclosure, and more particularly, detecting a fungus adhered to or entrapped within a chewable matrix according to a method of the present disclosure, wherein the chewable matrix was in contact with the oral cavity of subject (e.g., a masticated chewable matrix), and wherein the presence of the fungus in the saliva sample, and more particularly, the presence of the fungus adhered to or entrapped in the chewable matrix, is indicative of a fungal infection in the oral cavity of the subject.

The disclosure also provides for an in vitro method of determining the susceptibility of a mammal subject for developing a fungal infection in the oral cavity comprising detecting a fungus in a saliva sample collected following mastication of the chewable matrix of the present disclosure, and more particularly, detecting a fungus adhered to or entrapped within a chewable matrix according to a method of the present disclosure, wherein the chewable matrix was in contact with the oral cavity of the subject (e.g., a masticated chewable matrix), and wherein the presence of the fungus in the saliva sample, and more particularly, the presence of the fungus adhered to or entrapped within the chewable matrix, is indicative of increased susceptibility for developing a fungal infection in the oral cavity of the subject or developing a disease, condition or disorder associated with the presence of the fungus in the oral cavity of the subject.

The disclosure also provides for methods of detecting the presence of protozoa in the oral cavity of a subject, such as Entamoeba gingivalis and Trichomonas tenax. Optionally, the method may further comprise the step of diagnosing a protozoan infection in a mammalian subject, such as a human subject, by detecting the presence of the protozoa in a saliva sample collected following mastication of the chewable matrix of the present disclosure, and more particularly, detecting the presence of the protozoa adhered to or entrapped within a chewable matrix that was contacted with the oral cavity of the mammal (e.g., a masticated chewable matrix). The methods also may optionally comprise the step of informing a human subject of the presence of the protozoa or the diagnosis of a protozoan infection in the oral cavity.

The disclosure also provides for an in vitro method of diagnosing a protozoan infection in the oral cavity of a mammalian subject comprising detecting a protozoa in a saliva sample collected following mastication of the chewable matrix of the present disclosure, and more particularly, detecting a protozoa adhered to or entrapped within a chewable matrix according to a method of the present disclosure, wherein the chewable matrix was in contact with the oral cavity of subject (e.g. a masticated chewable matrix), and wherein the presence of the protozoa in the saliva sample, and more particularly, the presence of the protozoa adhered to or entrapped in the chewable matrix, is indicative of a protozoan infection in the oral cavity of the subject.

The disclosure also provides for an in vitro method of determining the susceptibility of a mammal subject for developing a protozoan infection in the oral cavity comprising detecting a protozoa in a saliva sample collected following mastication of the chewable matrix of the present disclosure, and more particularly, detecting a protozoa adhered to or entrapped within a chewable matrix according to a method of the present disclosure, wherein the chewable matrix was in contact with the oral cavity of the subject (e.g., a masticated chewable matrix), and wherein the presence of the protozoa in the saliva sample, and more particularly, the presence of the protozoa adhered to or entrapped within the matrix, is indicative of increased susceptibility for developing a protozoan infection in the oral cavity of the subject or developing a disease, condition or disorder associated with the presence of the protozoa in the oral cavity of the subject.

Furthermore, the methods of detecting a microorganism in a saliva sample collected following mastication of the chewable matrix of the present disclosure, and more particularly, detecting a microorganism adhered to or entrapped within a chewable matrix may be used to diagnose any disease related to a bacterial, viral, fungal or protozoan infection in the oral cavity. Exemplary conditions, diseases and disorders include chronic periodontitis, acute adult periodontitis, gingivitis such as acute necrotizing ulcerative gingivitis, Vincent angina, dental caries, herpesvirus infection, primary herpetic gingivostomatitis or oral herpes (cold sores and canker sores), genital herpes, varicella-zoster virus infection e.g. chicken pox or shingles, influenza, common cold, venereal disease, mononucleosis, coxsackievirus infection such as hand-foot-mouth disease, herpangina, acute lymphonodular pharyngitis, mumps, measles (reubeola), rubella (German measles), African Burkitt lymphoma, nasopharyngeal carcinoma, oral hairy leukoplakia, roseola infantum, Karposi sarcoma, Candidiasis, acute pseudomemranous candidosis (thrush), acute atrophic (erythematous) candidosis, chronic hyperplastic candidosis, chronic atrophic (erythematous) candidosis, aspergillosis, Scardovia wiggsiae infection; cryptococcosis, histoplasmosis, blastomycosis, paracoccidioidomycosis, and zygomycosis (mucormycosis).

Another aspect of the disclosure provides for methods of generating a microorganism profile of the oral cavity of a mammalian subject. The term “microorganism profile” refers to the presence or absence of at least one type of microorganism that is at least partially identified or characterized so that the presence or absence of the microorganism in any particular sample may be monitored. The term “microorganism profile” includes bacterial profiles, viral profiles, protozoan profiles, and fungal profiles and combinations thereof.

Thus, in one aspect, the present disclosure provides for methods of generating a microorganism profile of the oral cavity of a mammalian subject comprising a) contacting the oral cavity of the subject with a chewable matrix of the present disclosure, and b) detecting the presence or absence of at least one type of microorganism adhered to or entrapped within the chewable matrix after contact with the oral cavity of the subject, wherein the presence or absence of at least one microorganism determines the microorganism profile of the oral cavity of the subject. The microorganism may be contained on (e.g., “adhered to”) the chewable matrix or contained within (e.g., “entrapped within”) the folds of masticated chewable matrix.

In another aspect, the present disclosure provides for methods of generating a microorganism profile of the oral cavity of a mammalian subject comprising a) contacting the oral cavity of the subject with a chewable matrix of the present disclosure, and b) detecting the presence or absence of at least one type of microorganism in a saliva sample collected following mastication of the chewable matrix, wherein the presence or absence of at least one microorganism determines the microorganism profile of the oral cavity of the subject.

The microorganism profile may comprise information on at least one type of microorganism, at least two types of microorganisms, at least five types of microorganisms, at least 10 types of microorganisms, at least 20 types of microorganisms, at least 50 types of microorganisms, at least 100 types of microorganisms, or at least 500 types of microorganisms. The profile may comprise only one type of microorganism or multiple different types of microorganisms.

The methods of generating a microorganism profile may detect one or more of bacteria, virus, fungus or protozoa. For example, the bacteria detected to generate a microorganism profile or a bacterial profile include Streptococcus mutans, Streptococcus oralis, Actinomyces naeslundii, Streptococcus sanguinis, Porphyromomas gingivalis, Porphymomas intermedia, Bacteroides forsythus, Tanneraella forsythia, Campylobacter rectus, Eubacerium nodatum, Peptostreptococcus micros, Streptococcus intermedius, Aggregetibacter actinomycetemcomitans, Treponema denticola, Lactobacillus, Eikenella corrodens, Capnocytophaga gingivalis, Streptococcus gordonii, Veillonella parvula, Fusobacterium nucleatum, Scardovia wiggsiae, and Streptococcus sobrinus. Exemplary viruses that may be detected to generate a microorganism profile or a viral profile include Human Herpes Virus (HHV)-1 (also known as herpes simplex virus (HSV)-1), HHV-2 (HSV-2), HHV-3 (also known as varicella-zoster virus), HHV-4 (Epstein-Ban-virus), HHV-5 (cytomegalovirus), HHV-6, HHV-7, HHV-8, poliovirus, group A coxsackievirus, group B coxsackievirus, echovirus and Enterovirus 71 (EV-71), Human Papillomavirus family (HPV), e.g. HPV-16, HPV-18, HPV-33, HPV-35, mumps virus, Newcastle disease virus, human parainfluenza virus type 2, 4a and 4, Paramyxovirus, Rubivirus, human influenza virus A, e.g. H1N1 and H3N3, human influenza virus B, human influenza virus C, rhinovirus, and coronaviruses, which include severe acute respiratory syndrome coronavirus (SARS-CoV) and severe acute respiratory syndrome coronavirus 2 (SARS-CoV-2, which causes COVID-19 respiratory disease). Exemplary funguses that may be detected to generate a microorganism profile or a fungal profile include Candida e.g. Candida albicans, Aspergillus, Cryptococcus neoformans, Cryptococcus gattii, Histoplasma capsulatum, Blastomyces dermatitidis, Paracoccidioides brasiliensis, and Zygomycota. Exemplary protozoa that may be detected to generate a microorganism profile or a protozoan profile include Entamoeba gingivalis and Trichomonas tenax.

In one embodiment, the disclosure provides for “bacterial profiles” that comprise information on at least one type of bacteria, at least two types of bacteria, at least five types of bacteria, at least 10 types of bacteria, at least 20 types of bacteria, at least 50 types of bacteria, at least 100 types of bacteria or at least 500 types of bacteria. The term “bacterial profile” refers to the presence or absence of at least one type of bacteria that is at least partially identified or characterized so that the presence or absence of the bacteria in any particular sample may be monitored.

The disclosure also provides for microorganism profiles or viral profiles that comprises information on at least one type of virus, at least two types of virus, at least five types of virus, at least 10 types of virus, at least 20 types of virus, at least 50 types of virus, at least 100 types of virus and at least 500 types of virus.

In addition, the disclosure provides for microorganism profiles or fungal profiles that comprise information on at least one type of fungus (fungal profile), at least two types of fungi, at least five types of fungi, at least 10 types of fungi, at least 20 types of fungi, at least 50 types of fungi, at least 100 types of fungi or at least 500 types of fungi.

In addition, the disclosure provides for microorganism profiles or protozoan profiles that comprise information on at least one type of protozoa (protozoan profile), at least two types of protozoa, at least five types of protozoa, at least 10 types of protozoa, at least 20 types of protozoa, at least 50 types of protozoa, at least 100 types of protozoa or at least 500 types of protozoa.

These methods may optionally further comprise the steps of comparing the microorganism profile of the mammalian subject with a reference microorganism profile, wherein the reference microorganism profile is indicative of increased susceptibility for a disease, disorder or condition of the oral cavity and scoring the microorganism profile to determine whether the subject has increased susceptibility for a disease or disorder of the oral cavity. Further, these methods may optionally comprise the step of quantitating the microorganisms adhered to or entrapped within the chewable matrix.

The term “reference microorganism profile” refers to a microorganism profile generated for a known control or standard sample, such as a reference profile for a subject known to have increased susceptibility for a disease, disorder or condition of the oral cavity. For example, the microorganism profile may comprise one or more types of bacteria, one or more types of fungi, one or more types of protozoa, or one or more types of virus. Furthermore, the microorganism profile may comprise a combination of the microorganisms such as one or more types of bacteria, virus, fungi, and/or protozoa. The term “reference bacterial profile” refers to a bacterial profile generated for a known control or standard sample, such as a reference profile for a subject known to have increased susceptibility for a disease, disorder or condition of the oral cavity.

A microorganism profile or a bacterial profile of a subject may be associated with a reference microorganism profile or a reference bacterial profile when one or more of the microorganisms or bacteria in the respective reference profiles are present in the microorganism or bacterial profile of the subject. To determine if a subject microorganism or bacterial profile is associated with a reference microorganism profile or a reference bacterial profile, the profiles are scored to compare the subject microorganism or bacterial profile with the respective reference profiles.

Furthermore, the microorganism profiles of the present disclosure may be used to determine susceptibility of the subject for developing a disease, condition or disorder such as chronic periodontitis, acute adult periodontitis, gingivitis such as acute necrotizing ulcerative gingivitis, Vincent angina, dental caries, herpesvirus infection, primary herpetic gingivostomatitis or oral herpes (cold sores and canker sores), genital herpes, varicella-zoster virus infection e.g. chicken pox or shingles, influenza, common cold, venereal disease, mononucleosis, coxsackievirus infection such as hand-foot-mouth disease, herpangina, acute lymphonodular pharyngitis, mumps, measles (reubeola), rubella (German measles), African Burkitt lymphoma, nasopharyngeal carcinoma, oral hairy leukoplakia, roseola infantum, Karposi sarcoma, Candidiasis, acute pseudomemranous candidosis (thrush), acute atrophic (erythematous) candidosis, chronic hyperplastic candidosis, chronic atrophic (erythematous) candidosis, aspergillosis, cryptococcosis, histoplasmosis, blastomycosis, paracoccidioidomycosis, zygomycosis (mucormycosis), Scardovia wiggsiae infection, and respiratory diseases such as influenza and COVID-19.

Solvent Extraction of Saliva, Microorganisms, and Nucleic Acids from Chewable Matrix

Also provided herein are methods for extracting saliva, microorganisms, and/or nucleic acids from a chewable matrix, and methods for detecting and quantitating nucleic acids from microorganisms that are adhered to the external surface and/or entrapped within the internal surface of a chewable matrix that has been contacted with the oral cavity of a living organism, using an amplification method such as quantitative polymerase chain reaction (PCR). The number of microorganisms trapped in a chewable matrix of the present disclosure can be quantified and qualified.

Thus, in another aspect, the present disclosure is directed to a method of extracting saliva and/or microorganisms from a chewable matrix, and in particular a masticated chewable matrix. In certain non-limiting embodiments, the method comprises a) contacting a chewable matrix of the present disclosure (e.g., a masticated chewable matrix) with i) an organic solvent and ii) a buffer solution, and b) separating the organic solvent and buffer solution, wherein the saliva and/or microorganisms extracted from the chewable matrix are contained within the buffer solution. In another non-limiting embodiment of the method according to the present disclosure, microorganisms are extracted from the chewable matrix by contacting the chewable matrix with a solution comprising at least one selected from the group consisting of an organic solvent, a buffer, and water.

In another aspect, the present disclosure is directed to a method of extracting nucleic acids from a chewable matrix, and in particular a masticated chewable matrix. In certain non-limiting embodiments, the method comprises a) contacting a chewable matrix of the present disclosure (e.g., a masticated chewable matrix) with i) an organic solvent and ii) a buffer solution, and b) separating the organic solvent and buffer solution, wherein the nucleic acids extracted from the chewable matrix are contained within the buffer solution. In another non-limiting embodiment of the method according to the present disclosure, nucleic acids are extracted from the chewable matrix by contacting the chewable matrix with a solution comprising at least one selected from the group consisting of an organic solvent, a buffer, and water, and optionally subjecting to a vortex mixer to break up the chewable matrix.

These methods may further comprise a step of identifying the source of the nucleic acid extracted from the chewable matrix and/or a step of quantifying the nucleic acid extracted from the chewable matrix. The identifying step or quantifying step is carried out using an amplification method such as polymerase chain reaction, stand displacement, ligase chain reaction, helicase-dependent amplification, isothermal reverse transcription-thermophilic helicase-dependent amplification, rolling circle amplification, loop-mediated isothermal amplification or sequence based amplification.

In methods of the disclosure, the chewable matrix may be partially or completely dissolved by the organic solvent.

In some methods of the disclosure, the nucleic acids are extracted from an internal surface of the chewable matrix. The disclosure also provides for method of extracting nucleic acids from the chewable matrix wherein at least about 99% of the nucleic acids adhered to or entrapped within the chewable matrix are extracted, at least about 98% of the nucleic acids adhered to or entrapped within the chewable matrix are extracted, at least about 95% of the nucleic acids adhered to or entrapped within the chewable matrix are extracted, at least about 90% of the nucleic acids adhered to or entrapped within the chewable matrix are extracted, at least about 85% of the nucleic acids adhered to or entrapped within the chewable matrix are extracted, at least about 80% of the nucleic acids adhered to or entrapped within the chewable matrix are extracted, at least about 75% of the nucleic acids adhered to or entrapped within the chewable matrix are extracted, at least about 60% of the nucleic acids adhered to or entrapped within the chewable matrix are extracted, at least about 55% of the nucleic acids adhered to or entrapped within the chewable matrix are extracted or at least about 50% of the nucleic acids adhered to or entrapped within the chewable matrix are extracted.

In one embodiment, the disclosure provides for methods of extracting nucleic acids from microorganisms adhered to or entrapped within the chewable matrix, including microorganisms entrapped within the internal surface of the chewable matrix or microorganisms adhered to or entrapped within the external surface of the chewable matrix. The source of the nucleic acid may be any bacteria, virus, fungus or protozoa, including those set forth herein.

In another embodiment, the disclosure provides for methods of extracting nucleic acids from mammalian cells, such as human cells, canine cells, feline cells, murine cells, rat cells, bovine cells, equine cells, sheep cells, goat cells, primate cells, cells from aquatic mammals such as whales and dolphins and cells from other exotic mammals. For example, the disclosure provides for methods wherein the source of the nucleic acids is an epithelial cell, squamous cell, fibroblast, stem cell or keratocyte. The disclosure also provides for methods of extracting nucleic acids from cancer cells.

In another aspect, the present disclosure is directed to a method of detecting microorganisms adhered to or entrapped within a chewable matrix. Because the chewable matrix of the present disclosure includes organic polymers (i.e., polymers having a carbon base), the polymers will dissolve in organic solvents according to the methods of the present disclosure. Thus, in one aspect, the method comprises a) dissolving at least a portion of the chewable matrix in an organic solvent and a buffer solution; b) separating the organic solvent from the buffer solution, wherein nucleic acids extracted from the microorganisms are contained within the buffer solution, and c) amplifying the nucleic acids using at least one oligonucleotide primer specific for the microorganism. In another non-limiting embodiment of the method according to the present disclosure, microorganisms adhered to or entrapped within the chewable matrix following mastication are detected by (a) dissolving at least a portion of the chewable matrix in a solution comprising at least one selected from the group consisting of an organic solvent, a buffer, and water, (b) extracting nucleic acids from the microorganisms, and (c) amplifying the nucleic acids using at least one oligonucleotide primer specific for the microorganism.

According to certain non-limiting embodiments, the methods may be carried out with two or more primers, or with three or more primers. Suitable primers for use in this method are known in the art and include those described in U.S. Pat. No. 10,023,919, which is herein incorporated by reference in its entirety. Other suitable probes and primers suitable for use in the methods of the present disclosure include those for amplification of SARS-CoV-2. Such primers and probes are described at the website for Division of Viral Diseases, National Center for Immunization and Respiratory Diseases, Centers for Disease Control and Prevention, Atlanta, Ga., USA (https://www.cdc.gov/coronavirus/2019-ncov/lab/rt-per-panel-primer-probes.html). In this method, the microorganism detected may be adhered to, or entrapped within the external surface of the chewable matrix or entrapped within an internal surface of the chewable matrix. The disclosure contemplates carrying out the method of detecting microorganism adhered to or entrapped within a chewable matrix after the chewable matrix was in contact with a living organism and/or after the chewable matrix was masticated. The methods are carried out in vitro or outside of the living organism. These methods may be carried out for quality control purposes, e.g. working in the fields of food safety, food spoilage and fermentation. These methods may also be carried out for diagnosing an infection, disease, disorder or condition of the oral cavity of a subject, for diagnosing a respiratory infection, disease, disorder, or condition in a subject, for determining the susceptibility of a subject for developing a disease, disorder or condition, or for generating a microorganism profile of a subject.

The amplification step of these methods may be carried out using any method known in the art. For example, the nucleic acids of the microorganism adhered to or entrapped within the chewable matrix may be amplified using polymerase chain reaction, stand displacement, ligase chain reaction, helicase-dependent amplification, isothermal reverse transcription-thermophilic helicase-dependent amplification, rolling circle amplification, loop-mediated isothermal amplification, sequence based amplification or sequence based amplification.

These methods may further comprise a step of quantifying the number of microorganisms adhered to or entrapped within the chewable matrix. The identifying step or quantifying step is carried out using an amplification method such as polymerase chain reaction, stand displacement, ligase chain reaction, helicase-dependent amplification, isothermal reverse transcription-thermophilic helicase-dependent amplification, rolling circle amplification, loop-mediated isothermal amplification or sequence based amplification.

Organic Solvents

In embodiments of the present disclosure, the saliva, microorganisms, and/or nucleic acids are extracted from the chewable matrix using an organic solvent and a buffer solution. In one aspect, the methods of the disclosure comprise dissolving at least a portion of the chewable matrix in an organic solvent. Solvents are substances that are capable of dissolving or dispersing one or more other substances. Organic solvents are carbon-based solvents (i.e., they contain carbon in their molecular structure). Exemplary organic solvents that can be used in the methods of the disclosure include chloroform, xylene, toluene, 1,2 dichlorobenzene, hexane, tetrahydrofuran, dichloromethane or acetone. In one embodiment, the organic solvent is selected from the group consisting of chloroform, xylene, and toluene.

Buffers

The terms “buffered solution” or “buffer solution”, used interchangeably herein, refer to an aqueous solution that consists of a mixture of a weak acid and its conjugate base or a weak base and its conjugate acid, and which are used to keep the pH of a solution nearly constant. The methods of the disclosure can be carried out with any buffered solution that is conventional in the field of molecular biology and in which nucleic acids are stable. Exemplary buffers that may be used in any of the methods of the disclosure are set out in Table 1.

TABLE 1

| | | | Temp. | | |
|---|---|---|---|---|---|
| | | | Effect | | |
| Common | | Buffer | (dpH/dT | Mol. | |
| Name | pKₐ | Range | in 1/K) | Wt. | Full Compound Name |
| TRIS | 8.07 | 7.5-9.0 | −0.028 | 121.14 | tris(hydroxymethyl)methyl |
| | | | | | amine |
| Tricine | 8.05 | 7.4-8.8 | −0.021 | 179.2 | N- |
| | | | | | tris(hydroxymethyl)methyl |
| | | | | | glycine |
| TAPS | 8.43 | 7.7-9.1 | −0.018 | 243.3 | 3- |
| | | | | | {[tris(hydroxymethyl)meth- |
| | | | | | yl]amino}propanesulfonic acid |
| TAPSO | 7.635 | 7.0-8.2 | | 259.3 | 3-[N- |
| | | | | | Tris(hydroxymethyl)meth- |
| | | | | | ylamino]-2- |
| | | | | | hydroxypropanesulfonic acid |
| HEPES | 7.48 | 6.8-8.2 | −0.014 | 238.3 | 4-2-hydroxyethyl-1- |
| | | | | | piperazine ethanesulfonic |
| | | | | | acid |
| TES | 7.40 | 6.5-8.2 | −0.020 | 229.20 | 2- |
| | | | | | {[tris(hydroxymethyl)meth- |
| | | | | | yl]amino}ethanesulfonic acid |
| MOPS | 7.20 | 6.5-7.9 | −0.015 | 209.3 | 3-(N- |
| | | | | | morpholino)propanesulfonic |
| | | | | | acid |
| PIPES | 6.76 | 6.1-7.5 | −0.008 | 302.4 | piperazine-N,N′-bis(2- |
| | | | | | ethanesulfonic acid) |
| Cacodylate | 6.27 | 5.0-7.4 | | 138.0 | dimethylarsinic acid |
| SSC | 7.0 | 6.5-7.5 | | 189.1 | saline sodium citrate |
| MES | 6.15 | 5.5-6.7 | −0.011 | 195.2 | 2-(N- |
| | | | | | morpholino)ethanesulfonic |
| | | | | | acid |
| Succinic | 7.4 | 7.4-7.5 | | 118.09 | 2(R)-2- |
| acid | | | | | (methylamino)succinic acid |
| Bicine | 8.35 | 7.6-9.0 | −0.018 | 163.2 | N,N-bis(2- |
| | | | | | hydroxyethyl)glycine |

The term “Tris buffer” refers to a buffering solution comprising tris(hydroxymethyl)aminomethane. Tris buffer is also known as Tris base, Trizma, Trisamine, THAM, Tromethamine, Trometamol and Tromethane. In a particular embodiment, the buffered solution is Tris buffer.

The methods of the disclosure may also be carried out with buffer solutions that comprise Tris such as TAE buffer (Tris-acetate-EDTA, which is a solution containing Tris base, acetic acid and EDTA), or TBE buffer (Tris-borate-EDTA), which contains Tris base, boric acid and EDTA.

Alternatively, the methods of the disclosure may be carried out with lithium borate buffer (LB) which is a solution containing lithium hydroxide monohydrate and boric acid, or sodium borate buffer (SB).

Nucleic Acids

The term “nucleic acid,” “nucleic acid sequence” or “nucleic acid molecule” refers to a DNA or RNA sequence. The term encompasses molecules formed from any of the known base analogs of DNA and RNA such as, but not limited to 4 acetylcytosine, 8-hydroxy-N6-methyladenosine, aziridinyl-cytosine, pseudoisocytosine, 5-(carboxyhydroxylmethyl) uracil, 5-fluorouracil, 5-bromouracil, 5-carboxymethylaminomethyl-2-thiouracil, 5-carboxy-methylaminomethyluracil, dihydrouracil, inosine, N6-iso-pentenyladenine, 1-methyladenine, 1-methylpseudouracil, 1-methylguanine, 1-methylinosine, 2,2-dimethyl-guanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, S′-methoxycarbonyl-methyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The methods of the disclosure may detect and/or quantify naturally occurring or “native” nucleic acids or non-naturally occurring, non-native artificial, synthesized or artificial nucleic acids. The term “naturally occurring” or “native” when used in connection with biological materials such as nucleic acid molecules, polypeptides, host cells, and the like, refers to materials which are found in nature and are not manipulated by man.

The term “extracted nucleic acid” refers to a nucleic acid that (1) has been separated from at least about 50 percent of proteins, lipids, carbohydrates or other materials with which it is naturally found when total DNA is isolated from the source cells, (2) is not linked to all or a portion of a polynucleotide to which the “extracted nucleic acid” is linked in nature. Preferably, the isolated or extracted nucleic acid molecule of the present disclosure is substantially free from any other contaminating nucleic acid molecule(s) or other contaminants that are found in its natural environment.

Methods of Amplifying Nucleic Acids

The methods of the disclosure may comprise amplifying the nucleic acid that is extracted from a microorganism or cell present in a saliva sample following mastication of the chewable matrix of the present disclosure and in particular from a microorganism or cell adhered to or entrapped within the chewable matrix. The term “amplification method” refers to any method that increases the number of copies of a particular fragment of a nucleic acid sequence through replication of the fragment. These methods are typically carried out using thermal cycling instruments.

Polymerase chain reaction (PCR) is the most common nucleic acid amplification method using at least two primers and a DNA polymerase. Real-time PCR, also called quantitative PCR or qPCR, is a very sensitive method of amplifying and quantitation of nucleic acids. Real-time PCR detects the products of nucleic acid amplification during the course of the reaction and is generally considered to be more accurate than end-point PCR for determining initial target copy number.

Other nucleic acid amplification methods include strand displacement (SDA) which uses a stand-displacing DNA polymerase, isothermal amplification methods such as helicase-dependent amplification (HDA) and isothermal reverse transcription-thermophilic helicase-dependent amplification (RT-tHDA), rolling circle amplification (RCA) which is a unidirectional nucleic acid replication that can synthesize multiple copies of circular nucleic acids, loop-mediated isothermal amplification (LAMP) which uses a single temperature incubation for amplification, ligase chain reaction (LCA) which refers to the a nucleic acid amplification method that uses a thermostable DNA ligase and a thermostable DNA polymerase to amplify a target nucleic acid, nucleic acid sequence-based amplification (NASBA) is a one-step isothermal process for amplifying RNA and in vitro transcription. These methods may be carried out using techniques standard in the art. The present disclosure also contemplates using sequencing analysis to confirm the identity of DNA fragments amplified using PCR.

In the methods of the disclosure, PCR may be carried out using a “PCR reaction mixture” which is a mixture suitable for carrying out PCR. The PCR reaction mixture will contain a suitable amount of a thermostable DNA polymerase, a linear or circular template DNA, preferably double-stranded DNA, to be amplified, a pair of oligonucleotide primers such that one of the primers is configured for annealing to one strand of the template and the other primer is configured for annealing to the other or complementary strand of the template, ATP, suitable amounts of each of the four deoxyribonucleoside triphosphates (dNTPs), and buffers, salts such as MgCl2, preservatives, reducing agents, and water as may be required.

The oligonucleotide primers may be designed to specifically amplify a nucleic acid specific for genes of interest or a gene that identifies a particular microorganism. When designing the oligonucleotide primers, the length of a primer depends upon its (A+T) content, and the Tm of its partner. In addition, the primer should be complex enough to decrease the likelihood of the primer annealing to sequences other than the chosen target. The methods of the disclosure may utilize primers ranging in length from 10-30 nucleotides; preferably the primers will be 17 nucleotides in length. Generally, a 40%-60% G+C content is recommended for the primers, avoiding internal secondary structure and long stretches of any one base. In addition, primers should not anneal to regions of secondary structure (within the target) having a higher melting point than the primer.

The oligonucleotide primers may be universal primers that amplify a nucleic acid sequence that is present in all types of a particular microorganism such as universal primers that are specific for any type of bacteria, fungi, virus or protozoa, for example primers for bacterial ribosomal 16S rRNA gene. For example, universal primers may be specific for a particular type of bacteria such as universal primers specific for anaerobic bacteria, universal primers specific for aerobic bacteria, universal primer specific for gram-negative bacteria or universal primers specific for gram-positive microorganisms. Furthermore, the universal primers may be specific for a particular genus of microorganism, for example, universal primers that amplify a nucleic acid sequence specific for streptococci, or the universal primer may be specific for a particular species of microorganism, for example universal primers that amplify a nucleic acid sequence specific for Streptococcus mutans, universal primers may be specific for a particular strain of microorganism, for example universal primers that amplify a nucleic acid sequence specific for Streptococcus mutans MT8148.

Deoxyribonucleoside triphosphates (dNTPs) include 2′-deoxyadenosine 5′-triphosphate (dATP), 2′-deoxycytidine 5′-triphosphate (dCTP), 2′-deoxyguanosine 5′-triphosphate (dGTP), and 2′-deoxythymidine 5′-triphosphate (dTTP). Generally, the concentration of dNTP in the PCR reaction is about 200 μM. It is important to keep the four dNTP concentrations above the estimated Km of each dNTP (10 μM-15 μM) and balanced for best base incorporation. Lowering the concentrations of dNTP and magnesium ion by an equal molar concentration can improve fidelity. Modified dNTPs (dig-11-dUTP, 5-bromo-dUTP, inosine, biotin-11-dUTP, biotin-16-dUTP and 7-deaza dGTP) and 2′-deoxyuridine 5′-triphosphate (dUTP) also may be used.

Furthermore, in addition to PCR and other amplification methods, the extracted nucleic acids may be analyzed using any conventional assay to identify the source or the presence/expression of a particular sequence. These assays may be carried out in place of an amplification method or in combination with an amplification method. Exemplary assays include conventional and pulsed-field Southern blot, Northern blot, denaturing gradient gel electrophoresis, dot blot hybridization, slot blot hybridization, and microarrays. The chewable matrix, methods, and articles of kits described herein are not limited in this regard.

### Methods of Extracting Saliva and Microorganisms and Detecting and Quantifying Microorganisms Using Mechanical Force

Also disclosed are methods of extracting saliva and/or microorganisms from a chewable matrix, and more specifically, from a masticated chewable matrix, and methods for detecting and quantifying microorganisms, such as bacteria or viruses, present in a saliva sample collected following mastication of the chewable matrix, and in particular, adhered to or entrapped within the chewable matrix, using mechanical force in aqueous medium to remove the saliva and/or microorganisms from the chewable matrix. In some embodiments, the saliva and/or microorganisms may be removed from the chewable matrix by a vortex mixer, a centrifugal mixer such as SpeedMixer™, available from Synergy Devices Ltd in High Wycombe, United Kingdom, a shaker, a sonication system, or in other manners using mechanical force.

In one aspect, the present disclosure provides for methods of extracting saliva and/or microorganisms from a chewable matrix (e.g., a masticated chewable matrix), the method comprising sonicating the chewable matrix under conditions which remove the saliva and/or microorganisms from the chewable matrix and deposit the saliva and/or microorganisms in a suspension. In one embodiment, the method may optionally further comprise detecting microorganisms in the chewable matrix, the method further comprising contacting at least a portion of the suspension with a solid-support under conditions that promote colony formation, wherein the formation of a colony on the solid-support indicates the presence of a microorganism adhered to or entrapped within the chewable matrix. In this method, the chewable matrix can be contacted to an oral cavity of a subject (e.g., a mammal) prior to sonication such as the subject chewed the matrix (i.e., a masticated chewable matrix), or the microorganism attached to the chewable matrix outside of the oral cavity of a subject, such as by mechanical or robotic chewing stimulators. A “solid-support” is a matrix in which a microorganism, such as a bacteria, fungus, protozoa or virus can grow on such media comprising agar or another inert solidifying agent such as gelatin.

In another aspect, the present disclosure provides for methods of detecting a microorganism adhered to or entrapped within a chewable matrix (e.g., a masticated chewable matrix), the method comprising a) sonicating the chewable matrix under conditions which remove the microorganism from the chewable matrix and deposit the microorganism in a suspension, and b) contacting at least a portion of the suspension with a solid-support under conditions that promote colony formation, wherein the formation of a colony on the solid-support indicates the presence of a microorganism adhered to or entrapped within the chewable matrix. In this method, the chewable matrix can be contacted to an oral cavity of a subject (e.g., a mammal) prior to sonication such as the subject chewed the matrix (i.e., a masticated chewable matrix), or the microorganism attached to the chewable matrix outside of the oral cavity of mammal, such as by mechanical or robotic chewing stimulators.

Sonication refers to the act of applying sound energy, such as ultrasound, to agitate particles in a sample, e.g. sonicate the chewable matrix to remove a microorganism, such as a bacteria or virus attached thereto. Some sonicators utilize a probe that directly contacts the sample. Water bath based sonicators have ultrasound generating elements located below the tank which indirectly agitate the sample. The sonication must be carried out in standardized dimensions, such as sonicating chewable matrix that was formed to a particular shape using a mold. The mold may be any shape with standardized dimensions and may be of any material which only slightly or does not adhere to the chewable matrix such as polytetrafluoroethylene (TEFLON).

The methods of detecting a microorganism adhered to or entrapped within the chewable matrix may further comprise a step of quantifying the microorganisms adhered to or entrapped within the chewable matrix, wherein the number of colonies is indicative of the quantity of microorganisms adhered to or entrapped within the chewable matrix. The quantification may comprise generating a standard curve to determine the quantity of microorganisms adhered to or entrapped within the chewable matrix. The combination of sonicating the chewable matrix and the generation of the standard curve allows for using the number of microorganisms on the surface of the chewable matrix to extrapolate the total number of microorganism adhered to or entrapped within the chewable matrix.

In another aspect, the disclosure provides for methods of detecting the presence of a microorganism in the oral cavity of a subject (e.g., a mammal) comprising a) sonicating a chewable matrix of the present disclosure that was in contact with the oral cavity of the subject under conditions which remove the microorganism from the chewable matrix and deposit microorganism in a suspension, b) contacting at least a portion of the suspension with a solid-support under conditions that promote colony formation, wherein the formation of a colony on the solid-support indicates the presence of a microorganism in the oral cavity of the subject. The sonication may be carried out in standardized dimensions, such as sonicating the chewable matrix that was formed to a particular shape using a mold.

The methods of detecting the presence of a microorganism in the oral cavity of a subject may further comprise the step of quantifying the microorganism contained within the oral cavity of the subject, wherein the number of colonies is indicative of the quantity of microorganisms within the oral cavity of the subject. The quantification may comprise generating a standard curve to determine the quantity of microorganisms within the oral cavity of the subject.

In another aspect, the disclosure provides for methods of quantitating microorganisms adhered to or entrapped within a chewable matrix of the present disclosure, the method comprising a) sonicating the chewable matrix under conditions which remove the microorganisms from the chewable matrix and deposit the microorganisms in a suspension, b) contacting at least a portion of the suspension with a solid-support under conditions that promote colony formation, and c) quantitating the colonies formed on the solid-support, wherein the number of colonies indicates the quantity of microorganisms adhered to or entrapped within the chewable matrix. The methods of quantitating microorganisms adhered to or entrapped within the chewable matrix may further comprise the step of generating a standard curve to determine the quantity of microorganisms attached to the chewable matrix. The sonication may be carried out in standardized dimensions, such as sonicating chewable matrix that was formed to a particular shape using a mold.

Any of the above-described methods may further comprise the step of identifying the microorganism. These microorganisms include bacteria, virus, fungus and protozoa.

In addition, any of the above-described methods of the disclosure may further comprise the step of diagnosing a microorganism infection in a mammalian subject wherein the chewable matrix was in contact with the oral cavity of the subject and wherein the presence of microorganisms adhered to or entrapped within the chewable matrix is indicative of a microorganism infection in the subject.

Thus, the present disclosure provides for methods of detecting and quantitating microorganisms adhered to or entrapped within a masticated chewable matrix of the present disclosure using sonication to remove the microorganisms from the chewable matrix and depositing the microorganisms on a solid-support under conditions that promote colony growth, wherein the formation of a colony on the solid-support indicates the presence of microorganisms adhered to or entrapped within the chewable matrix. The number of colonies formed allows for the generation of a standard calibration curve. This method consistently determines the number of microorganisms adhered to or entrapped within the chewable matrix independent of type of microorganism tested. As sonication can only release microorganisms attached to the surface of the chewable matrix, the number of microorganisms retrieved is roughly 1 log-unit less than chewed in.

The use of the calibration curve allows for the calculation of the number of microorganisms adhered to or entrapped within the chewable matrix. Specifically, the combination of sonicating the chewable matrix and the generation of the standard curve allows for using the number of microorganisms on the surface of the chewable matrix to extrapolate the total number of microorganisms adhered to or entrapped within the chewable matrix. One method of generating the calibration curve is to sonicate the chewable matrix, wherein the chewable matrix is in standardized dimensions, such as a chewable matrix that was formed to a particular shape using a mold. This mold provides a fixed surface area for a given weight of chewable matrix, so that the number of microorganisms attached to the surface of the chewable matrix will serve as a means to calculate the number of microorganisms within the volume of chewable matrix.

The mold may be any shape with standardized dimensions, for example the mold is a rectangle with dimensions of 15×15×4 mm or a cube with the dimensions 12³ mm³. The mold may be of any material, such as polytetrafluoroethylene (PTFE) also known as TEFLON or DYNEON, polyvinylindene fluoride (PVDF) also known as KYNAR, HYLAR and SOLEF; polyoxymethylene (POM) also known as DELRIN, CELCON, DURACON and HOSTAFORM; ethylene tetrafluorthylene (ETFE) also known as TEFZEL, polyamide-imides also known TORLON; perfluoroalkoxy (PFA) or fluorinated ethylene propylene (FEP).

The consistency of the calibration method allows for accurate testing regardless of the type of microorganisms adhered to or entrapped within the chewable matrix. The disclosure also contemplates carrying out the methods using artificial or robotic chewing stimulators that reproduce mandibular movements exerted during mastication. Although the recovery may be low (the majority may be still trapped in the chewable matrix), the calibration curve accounts for these losses and makes the estimation of the amount of microorganism that is trapped more accurate. Furthermore, the method of the disclosure only enumerates live microorganism that are cultivable. When the chewable matrix contacts the oral cavity of mammal, microorganisms such as bacteria adhere to the chewable matrix. Therefore, the methods of the disclosure can be used to detect the presence of a microorganism in the oral cavity of a mammal. The knowledge of the presence of certain microorganisms, such as bacteria, in an oral cavity can be used to diagnose an infection or other diseases or disorders of the oral cavity. In addition, the presence of certain microorganisms, such as certain bacteria, in the oral cavity may allow for the determination of the risk or susceptibility of a mammal for developing an infection disease or disorder of the oral cavity.

Microorganism Colonies

In one embodiment of the disclosure, the suspension of microorganism, such as bacteria, virus, protozoa or fungi, is deposited on a solid support such as media comprising agar or another inert solidifying agent such as gelatin. The degree of solidification can also vary, with stiff agar being preferred to inhibit “swarming” and semi-solid or “sloppy” agar being used to observe other characteristics. Before utilization, such medium is preferably sterile.

The solid support may be a slant, stable or petri dish comprising the solid media. The solid medium has physical structure which allows bacteria to grow in physically informative or useful ways such as in colonies or in streaks. The term “colony” refers to the pile or mass of cells or organisms growing on or in solid medium. The disclosure contemplates that the solid media may comprise a colorimetric indicator, be selective media or differentiation media.

The nutrient media utilized in the methods of the disclosure is any liquid or solid preparation suitable for the growth, maintenance, storage, differential, isolation and/or identification of bacteria. These include those utilized for the initiation of a culture (or subculture), for enrichment, or for diagnostic (identification) tests of various organisms. These are tests in which the identity of a given organism may be deduced from the characteristics of its growth in or on particular media.

Selective media refers to media designed to suppress the growth of some bacteria while allowing the growth of other bacteria. For example, MacConkey agar selects against gram-positive bacteria, eosin-methylene blue agar selects against gram-positive bacteria and phenylethyl alcohol selects against gram-negative bacteria. Differential media allow the growth of more than one bacteria of interest but with morphologically distinguishable colonies. For example, mannitol salts agar distinguishes mannitol fermentation in the color yellow, blood agar distinguishes various kinds of hemolysis, MacConkey agar distinguishes lactose fermentation in the color yellow, and eosin-methylene blue agar allows for various kinds of differentiation.

For anaerobic bacteria, the culture requires an oxygen-free gaseous above the surface of the medium and a medium free from dissolved oxygen. Even under these conditions, some anaerobes may not grow unless the medium has been pre-reduced, i.e. poised at or below a particular redox potential. Consequently, reducing agents, such as those containing sulfhydryl groups (e.g., H₂S, cysteine, thioglycollate) may also be included in the medium composition. Examples of commonly available medium being suitable for use for the selective growth of anaerobes is in the present disclosure, include, but are not limited to, Brain Heart Infusion, Brucella, CDC Anaerobe, Nutrient, Schaedler, Thioglycollate or Trypticase Soy. These are in both broth or agar form.

Additionally, the medium may be made anaerobic in an anaerobic jar, chamber or bag. An anaerobic jar is a container used for the incubation of materials (e.g. inoculated media) in the absence of oxygen or, in general, under gaseous conditions other than atmospheric. These are commonly known under the designations “Brewer Jar”, “Gaspak”, “McIntosh and Filde's Anaerobic Jar”, etc.

The culture medium is inoculated with a sample of the suspension of the microorganism removed from the chewable matrix. The suspension may be diluted prior to inoculating the culture medium or being applied to the solid support. In certain embodiments, the sample may be serially diluted and the serial dilutions used to inoculate a plurality of culture media in order to obtain a more precise enumeration of bacteria in the original sample.

In another embodiment of the present disclosure, the methods comprise growing the bacteria removed from the chewable matrix and broth media may be inoculated with the diluted suspension of bacteria removed from the chewable matrix. Broth media refers to media lacking solidifying matrix. The inoculated culture medium is incubated under conditions that permit the growth of bacteria. The broth media may allow for the detection of the type of media. For example, the media comprises a colorimetric indicator, selective media or differential bacteria.

In some embodiments of the method of the present disclosure, the number of microorganisms present in the sample may be enumerated. For example, one may enumerate the number of bacteria in a sample by selecting a form of culture medium that permits the formation of colonies. After the culture medium is inoculated and incubated under conditions that permit colonies of the bacteria to form, the colonies may be counted. Colonies may be counted by counting the detectable signal generated by reaction of the indicator and phosphatase produced by the bacteria. In embodiments in which the phosphatase substrate indicators 5-bromo-6-chloro-3-indolylphosphate or 6-chloro-3-indolylphosphate are employed, the detectable signal may include red to red-violet colored colonies. Thus, in certain embodiments, enumeration of microorganism includes merely counting the particular color colonies such as the red to red-violet colonies under phosphate conditions. Suitable detectable signals include but are not limited to a chemiluminescent signal, a fluorescent signal, or a change in electrical conductivity. In some embodiments, detection of the detectable signal may be accomplished manually, while in other embodiments detection of the detectable signal may require specialized detection instrumentation known to those of ordinary skill in the art. The method used to enumerate microorganisms in a particular sample may depend, at least in part, on the type of detectable signal used in the method of the present disclosure.

In addition to quantifying the microorganisms removed from the chewable matrix, the method of the disclosure may be used to identify the microorganism removed from the chewable matrix. The identification may be based on taxonomic principles based on morphologic and metabolic characteristics. Colony morphology may be analyzed to identify the type of bacteria removed from the chewable matrix. The term “colony morphology” refers to the visual characteristics of a colony. Colonies that differ in appearance are typically different bacterial family, genus, species, strain, serotype or serogroup. Colony morphology is evaluated based on the colony shape, margin, color, surface features, elevation, light transmission or pigmentation to name a few.

There are many different tests known in the art which distinguish microorganisms. Molecular biology techniques used for the identification of specific genes or gene segments of known bacteria include PCR, northern blotting, conventional and pulsed-field Southern blot, denaturing gradient gel electrophoresis, microarrays, slot blotting or dot blotting using polynucleotide sequences specific for a specific family, genus, species, strain, serotype or serogroup.

For example, determining the sequence of the 16S rRNA and other regions within the bacterial genome may be used to identify a type of bacteria. Analytical chemistry techniques such as determining fatty acid profiling, carbohydrate profiling and ubiquinone profiling, characterization of secreted metabolic products such as volatile alcohols and short chain fatty acids are also used to identify a type of bacteria.

In another embodiment of the disclosure, the methods comprise depositing the suspension of virus on a confluent monolayer of cells affixed within a solid support such as media comprising agar, carboxymethyl cellulose or another inert solidifying agent such as gelatin as described above. The viral colonies are also known as “viral plaque.” The viral plaque is formed when the virus infects a cell within the fixed monolayer. The virus will lyse the cell and the infection will spread to other cells. The infected cells create a plaque which can be visually seen with a microscope. Assays for determining a quantity of virus are well known in the art such as those that utilize immunofluorescence, colorimetric measurement, proteins and hemagglutination, see e.g., Kaufman & Kabelitz, Methods of Microbiology, Vol. 32, Immunology of Infection, Academic Press, 2002.

In addition to quantifying the virus removed from the chewable matrix, the methods of the disclosure may be used to identify the virus removed from the chewable matrix. Molecular biology techniques used for the identification of specific genes or gene segments of known viruses include PCR, northern blotting, conventional and pulsed-field Southern blot, denaturing gradient gel electrophoresis, microarrays, slot blotting or dot blotting using polynucleotide sequences specific for a specific family, genus, species, strain, serotype or serogroup. Microscopy may be also used to identify morphological characteristics of the virus, such as the observing the size, shape or other distinct morphological features of the virus.

### Chewable Matrix

The present disclosure provides a chewable matrix that can be used in the methods disclosed herein. The chewable matrix of the present disclosure comprises a water-insoluble portion and optionally, a water-soluble portion. In particular, the chewable matrix comprises high levels of low molecular weight polyvinyl acetate (PVAc). It is believed that inclusion of high levels of low molecular weight polyvinyl acetate in the chewable matrix improves the ability of the chewable matrix to hydrate the oral cavity upon mastication and to absorb saliva. Additionally, unlike traditional polymer-based chewable confectionery products, the chewable matrix of the present disclosure contains minimal amounts of flavor and sweeteners, which may interfere with collection of saliva and/or microorganism collection. The chewable matrix of the present disclosure is also softer, more easily hydrated, and less cohesive than conventional polymer-based chewable confectionery products.

Thus, in one aspect, the chewable matrix comprises high levels of low molecular weight PVAc. The molecular weight of the PVAc used in the chewable matrix ranges from an average of about 6,000 daltons to an average of about 40,000 daltons. Preferably, the average molecular weight of the low molecular weight PVAc is less than 17,000 daltons, including in the range of from about 12,000 daltons to about 16,000 daltons. Unless otherwise indicated all molecular weights in the current disclosure are weight average molecular weight. The low molecular weight PVAc is used in the chewable matrix in amounts up to about 99% by weight of the matrix. In one embodiment, the amount of low molecular weight PVAc present is in the range from 5% to about 95% by weight of the matrix, or from 5% to about 40% by weight of the matrix, or from about 10% to about 20% by weight of the matrix, or from about 13% to about 18% by weight of the matrix. In one embodiment, the low molecular weight PVAc is present in the chewable matrix in an amount of about 15% to 16% by weight of the matrix. In one embodiment, the amount of low molecular weight PVAc is present at about 34% by weight of the matrix. In another embodiment, the amount of low molecular weight PVAc is present in an amount of from about 40% to about 99% by weight of the matrix, or from about 60% to about 98% by weight of the matrix, or from about 75% to about 95% by weight of the matrix. One PVAc suitable for use in the chewable matrix of the present disclosure is Vinnapas B15 Spezial, available from Wacker in Burghausen, Germany.

In addition to low molecular weight PVAc previously described, the chewable matrix of the present disclosure optionally further comprises at least one elastomer. Suitable elastomers for use in the chewable matrix of the present disclosure include any elastomers known in the art for inclusion in a chewable polymer-based confectionery product. Elastomers provide the rubbery, cohesive nature to the product which varies depending on this ingredient's chemical structure and how it is compounded with other ingredients. Elastomers suitable for use in the chewable matrix of the present disclosure include butadiene-styrene copolymers (SBR), isobutylene-isoprene copolymers (Butyl rubber), polybutadiene, low, medium, or high molecular weight polyisobutylene, and vinyl polymeric materials (polyethylene, vinyl acetate/vinyl laurate, vinyl acetate/vinyl stearate, ethylene/vinyl acetate) or mixtures thereof. The elastomer may be included in the chewable matrix in an amount of from about 0% to about 15% by weight of the matrix, including from about 1% to about 15% by weight of the matrix, or from about 4% to about 8% by weight of the matrix.

In one embodiment, the total polymer content of the chewable matrix (e.g., elastomer, PVAc, other polymer, etc.) is from about 20% to about 33% by weight of the matrix.

In one embodiment, the elastomer is a high molecular weight polyisobutylene. High molecular weight polyisobutylene provides a cohesive property and is believed to reduce the cold flow property of a cud formed following mastication of the chewable matrix. The high molecular weight polyisobutylene also improves the softness of a chewable matrix for improved organoleptic quality. The average molecular weight of the high molecular weight polyisobutylene used in chewable matrix ranges from about 200,000 daltons to about 600,000 daltons. Preferably, the average molecular weight of high molecular weight polyisobutylene used in the chewable matrix is about 400,000 daltons. The amount of high molecular weight polyisobutylene in the inventive chewable matrix ranges from 0% to about 15%, by weight of the matrix, including from about 5% to about 15% by weight of the matrix. In one embodiment, the amount of high molecular weight polyisobutylene is present in the amount of about 8% by weight of the matrix. A high molecular weight polyisobutylene suitable for use in the present disclosure is OPANOL 50 SF, available from BASF in Ludwigshafen, Germany.

Alternately, in some embodiments, the chewable matrix may be essentially free of high molecular weight elastomers, such as high molecular weight polyisobutylene. As used herein, the term “high molecular weight elastomers” refers to elastomers having a molecular weight of greater than 100,000 daltons, and more typically, a molecular weight of 200,000 daltons or greater. For purposes of the description of the present disclosure, being essentially free of high molecular weight elastomers can mean that the optional use of such elastomers at levels of about 0% to about 5% by weight of the chewable matrix is acceptable. In an embodiment of the present disclosure, the chewable matrix is free of high molecular weight elastomers (i.e., contains 0% by weight of the matrix).

In one embodiment, the chewable elastomer comprises a low molecular weight PVAc, such as described herein, and an elastomer having a molecular weight of 100,000 daltons or less (also referred to here as a “low molecular weight elastomer”). Examples of suitable low molecular weight elastomers include, but are not limited to, low molecular weight polyisobutylene (e.g., having a molecular weight of 100,000 daltons or less) and low molecular weight butyl rubber (e.g., having a molecular weight of 100,000 daltons or less). In one such embodiment, the low molecular weight PVAc has a molecular weight of 17,000 daltons or less. In one embodiment, the chewable matrix comprises low molecular weight PVAc, at least one low molecular weight elastomer, and is essentially free of high molecular weight elastomers. In one embodiment, the chewable matrix comprises low molecular weight PVAc, at least one low molecular weight elastomer, and is free of high molecular weight elastomers (i.e., comprises 0% by weight of the matrix of high molecular weight elastomers). In one embodiment, the chewable matrix comprises low molecular weight PVAc in an amount of from about 13% to about 35% by weight of the matrix, and low molecular weight elastomer (such as low molecular weight polyisobutylene and/or low molecular weight butyl rubber), in an amount of from about 3% to about 17% by weight of the matrix.

In one embodiment, the chewable matrix of the present disclosure is essentially free of filler. The filler may be a non-silica filler or a silica filler. In one embodiment, the chewable matrix is essentially free of non-silica filler. For purposes of the description of the present disclosure, being essentially free of filler (or essentially free of non-silica filler) can mean that the optional use of filler (or non-silica filler) at levels of about 0% to about 5% by weight of the chewable matrix is acceptable. It is believed that this increases the viscosity and minimizes the plasticization of the chewable matrix upon chewing. In an embodiment of the present disclosure, the chewable matrix is free of filler (i.e., contains 0% by weight of filler). In one embodiment, the chewable matrix is free of non-silica filler (i.e., contains 0% by weight of non-silica filler). In other non-limiting embodiments, the chewable matrix is not free of filler.

Amorphous silica may optionally be added to the chewable matrix because silica has low oil absorption properties as compared to non-silica fillers. One amorphous silica which may be used in the present disclosure has an average particle size of 16 μm, pH of about 7, oil absorption of about 55 g/100 g and Perspex abrasion value of about 35. The specifications of the silica used is not believed to be critical, but specifications of silicas known to be operable are herein disclosed. The silica in the present disclosure may have a range of average particle size of 4.5 to 18 μm. The amount of amorphous silica used in the present disclosure ranges from about 0% to about 15% by weight of the chewable matrix, or from about 2% to about 15% by weight of the chewable matrix. In one embodiment, the amount of amorphous silica used in the chewable matrix is about 5% by weight of the chewable matrix. These levels include any moisture, typically 2% to 4%, that may be present on commercially available silicas. The addition of amorphous silica may improve the organoleptic quality of the chewable matrix and has low oil absorption properties. The amorphous silica used in the present disclosure is preferably DH338 and is available from INEOS Silicas Inc., in Warrington, England. In another embodiment, the chewable matrix is essentially free of amorphous silica. In one embodiment, the chewable matrix is free of amorphous silica.

Non-silica fillers, which may be used at levels up to 5% in the chewable matrix, may be selected from carbonate or precipitated carbonate types, such as magnesium and calcium carbonate, ground limestone, silicate types such as magnesium and aluminum silicate, clay alumina, talc, titanium dioxide, mono-, di- and tricalcium phosphate, and mixtures thereof. Non-silica fillers which may be used as a filler to levels up to 5% in the chewable matrix are most typically calcium carbonate and talc. Talc filler may be used in the chewable matrix that may come in contact with or employ acid flavors or provide an acidic environment needed to prevent degradation of an artificial sweetener.

Mean particle size for calcium carbonate and talc fillers typically range from about 0.1 micron to about 15 microns. More preferably, the optional fillers used preferably have a mean particle size range from about 0.4 to about 14 microns and are calcium carbonate and talc.

In other embodiments, the chewable matrix may comprise filler in amounts greater than 5% by weight of the matrix. Any of the silica or non-silica fillers described herein may be included in these amounts.

The chewable matrix of the present disclosure may optionally include plasticizers. Plasticizers used in the chewable matrix may include water, triacetin, glycerin, propylene glycol, butyl lactate, triethyl citrate, 2-ethylhexyl-s-lactate, propylene glycol laurate, propylene glycol monolaurate, sorbitan monolaurate, propylene glycol dicaprylocaprate, bis(2-ethylhexyl)adipate, polypropylene glycol dibenzoate, dipropylene glycol dibenzoate, butyl phthalyl butyl glycolate, di-n-butyl malate, tri(2-ethylhexyl) citrate dibutyl sebacate, medium chain triglyceride, mono-, di- and triglycerides of fatty acids, terpene resins derived from alpha-pinene, beta-pinene or d-limonene, triglycerides of non-hydrogenated, partially hydrogenated and fully hydrogenated vegetable oil, including cottonseed oil, soybean oil, palm oil, palm kernel oil, coconut oil, safflower oil, tallow oil, cocoa butter, unsaturated oils that contain, as one or more of their constituent groups, fatty acids of carbon chain length of from 6 to 18, monoglycerides, diglycerides, acetylated monoglycerides, distilled mono-, and di-glycerides and lecithin may, from their manufacturing processing, contain triglyceride levels less than 2 percent by weight. Mono- and diglycerides may be considered as being of the same family as fats. In one embodiment, the chewable matrix may include plasticizers in an amount of from 0% to about 40% by weight of the matrix, including from about 0.5% to about 40% by weight of the matrix, or from about 0% to about 10% by weight of the matrix, or from about 0.5% to about 10% by weight of the matrix.

In one particular embodiment, the chewable matrix comprises low molecular weight PVAc and at least one plasticizer. In one such embodiment, the chewable matrix comprises low molecular weight PVAc in an amount of up to about 99% by weight of the matrix, including in an amount of from about 5% to about 95% of the matrix, or from about 35% to about 95% by weight of the matrix, or from about 40% to about 99% by weight of the matrix, or from about 60% to about 98% by weight of the matrix, or from about 75% to about 95% by weight of the matrix. In one embodiment, the matrix comprises plasticizer in an amount of from about 0.5% to about 15% by weight of the matrix. In one such embodiment, the plasticizer is selected from the group consisting of water, triacetin, an acetylated monoglyceride, glycerin, propylene glycol, butyl lactate, triethyl citrate, 2-ethylhexyl-s-lactate, propylene glycol laurate, propylene glycol monolaurate, sorbitan monolaurate, propylene glycol dicaprylocaprate, bis(2-ethylhexyl)adipate, polypropylene glycol dibenzoate, dipropylene glycol dibenzoate, butyl phthalyl butyl glycolate, di-n-butyl malate, tri(2-ethylhexyl) citrate and dibutyl sebacate, and combinations thereof. In one embodiment, the plasticizer is selected from the group consisting of water, triacetin, an acetylated monoglyceride. In such an embodiment, the chewable matrix may be essentially free of (i.e., contain less than 5% by weight of the matrix) or free of (i.e., contain 0% by weight of the matrix) elastomers. In one embodiment, the chewable matrix is free of or essentially free of elastomers. In one embodiment, the chewable matrix comprises bulking agents in an amount of 60% or less by weight of the matrix. In one embodiment, the chewable matrix is free of flavoring agents.

The chewable matrix of the present disclosure may optionally include emulsifiers. Emulsifiers, which also sometimes have plasticizing properties, used in chewable matrix may be selected from the following, glycerol mono and distearate, lecithin, mono and di-glycerides of fatty acids, triacetin, acetylated monoglyceride, polyglycerol esters, glycerol triacetate and carbohydrate polyesters, or combinations thereof. In one embodiment, the chewable matrix is free of soy lecithin.

Other optional ingredients such as antioxidants may also be used in the chewable matrix. Antioxidants prolong shelf-life and storage of the chewable matrix, or its components. Antioxidants suitable for use in the chewable matrix of the present disclosure include butylated hydroxyanisole (BHA), butylated hydroxytoluene (BHT), beta-carotenes, tocopherols, acidulants such as vitamin C, propyl gallate, and other synthetic and natural types, or mixtures thereof. Preferably, the antioxidants used in the chewable matrix are butylated hydroxyanisole (BHA), butylated hydroxytoluene (BHT), tocopherols, or mixtures thereof.

In one embodiment, the chewable matrix is essentially free of waxes.

In another embodiment, the chewable matrix may optionally comprise synthetic waxes. Synthetic waxes are produced by means atypical of petroleum wax production and thus are not considered petroleum wax. These synthetic waxes may be used in accordance with the present disclosure and may be included optionally in the chewable matrix. The synthetic waxes may include waxes containing branched alkanes and copolymerized with monomers such as, but not limited to, polypropylene and polyethylene and Fischer-Tropsch type waxes. Polyethylene wax is not in the same category as polyethylene, a polymer of ethylene monomers. Rather, polyethylene wax is a synthetic wax containing alkane units of varying lengths having attached thereto ethylene monomers.

In one embodiment, the chewable matrix may optionally comprise elastomer plasticizers. Elastomer plasticizers vary the firmness of the matrix. The chewable matrix is preferably free of ester gums. The plasticizers used are synthetic elastomer plasticizers such as terpene resins derived from alpha-pinene, beta-pinene and/or d-limonene and mixtures thereof.

The elastomer plasticizers used may be of one type or of combinations of more than one. Typically, the ratios of one to the other are dependent on each respective softening point, on each effect on flavor release, and on each respective degree of tack they cause to the matrix. The preferred elastomer plasticizers for use in the chewable matrix of the present disclosure are terpene resins. The approximate amount of terpene resin used in the chewable matrix (when included) is from 0% to about 44% by weight of the chewable matrix, including from about 2% to about 20% by weight of the chewable matrix, or from about 5% to about 25% by weight of the chewable matrix, or from about 10% to about 35% by weight of the chewable matrix.

In some embodiments, the chewable matrix may additionally comprise water-soluble ingredients. The water-soluble portion of the chewable matrix may optionally comprise softeners, sweeteners, flavoring agents, and combinations thereof. The sweeteners often function also as bulking agents in the chewable matrix. The total amount of bulking agents in the chewable matrix may be about 60% or less, including from about 5% to about 50%, or from about 5% to about 20%, or from about 10% to about 15% by weight of the matrix. In one embodiment, the chewable matrix is essentially free of bulking agents (i.e., contains 5% or less by weight of the matrix). In another embodiment, the chewable matrix is free of bulking agents (e.g., sweeteners) (i.e., contains 0% by weight of the matrix).

Sugar sweeteners generally include saccharide-containing components commonly known in the confectionery art which comprise, but are not limited to, sucrose, dextrose, maltose, dextrin, dried invert sugar, fructose, levulose, galactose, corn syrup solids and the like, alone or in any combination.

The chewable matrix can also include sugarless sweeteners. Generally, sugarless sweeteners include components with sweetening characteristics but which are devoid of the commonly known sugars and comprise, but are not limited to, sugar alcohols such as sorbitol, mannitol, erythritol, isomalt, xylitol, hydrogenated starch hydrolysates, maltitol and the like, alone or in any combination.

High intensity artificial sweeteners can also be included in the chewable matrix, alone or in combination, with the above. Preferred sweeteners include, but are not limited to, sucralose, aspartame, NAPM derivatives such as neotame, salts of acesulfame, alitame, saccharin and its salts, cyclamic acid and its salts, glycyrrhizinate, dihydrochalcones, thaumatin, monellin, and the like, alone or in combination. In order to provide longer lasting sweetness and flavor perception, it may be desirable to encapsulate or otherwise control the release of at least a portion of the artificial sweetener. Such techniques as wet granulation, wax granulation, spray drying, spray chilling, fluid bed coating, coacervation, and fiber extension may be used to achieve the desired release characteristics.

De-oiled lecithin, such as de-oiled soy lecithin, may optionally be included in the chewable matrix. De-oiled lecithin provides low oil absorption qualities. The de-oiled lecithin is also a process aid in the production of the chewable matrix. De-oiled lecithin, in addition, retains moisture and absorbs moisture readily. The lecithin may be a powdered de-oiled lecithin and mixed with the bulking agent (sugar, sorbitol etc.) before adding to the mixer. The de-oiled lecithin may be used in the chewable matrix in amounts of about 0% to about 50% by weight of the matrix. Preferably, the de-oiled soy lecithin is used in amounts of about 13% to about 50%, 20% to about 50%, from about 17% to about 40%, or from about 30% to about 45% by weight of the matrix. In one embodiment, the chewable matrix comprises de-oiled lecithin (e.g., de-oiled soy lecithin) in an amount of about 40% by weight of the matrix. The lecithin may also be used in an encapsulated form. Lecithin substitutes may also be used to provide the same advantages described herein. Specific fractions of lecithin purifications may also be used to provide the same advantages described herein. The powdered lecithin used in the present disclosure may be EMULPUR IP and EMULGUM from Degussa in Hamburg Germany.

Softeners may be added to the chewable matrix in order to optimize the chewability and mouth feel of the matrix. Softeners typically constitute from approximately 0.5% to about 25.0% by weight of the matrix. Softeners contemplated for use in the chewable matrix include glycerin, lecithin and combinations thereof. Further, aqueous sweetener solutions such as those containing sorbitol, hydrogenated starch hydrolysates, corn syrup and combinations thereof may be used as softeners and bulking agents. Sugar-free formulations are also typical.

Glycerin may optionally be included in the chewable matrix in an amount of from 0% to about 15% by weight of the matrix, including from 0% to about 10% by weight of the matrix, or from about 3% to about 15% by weight of the matrix, or from about 3% to about 7% by weight of the matrix. In one embodiment, the matrix comprises glycerin in an amount of about 5% by weight of the matrix.

The chewable matrix of the present disclosure may further optionally comprise flavorants and/or colorants. Flavorants and colorants impart characteristics or remove or mask undesired characteristics.

A flavoring agent may optionally be present in the chewable matrix in an amount within the range of from approximately 0.1 to about 10.0 weight percent, and preferably from approximately 0.5 to about 3.0 weight percent of the matrix. In other embodiments, the chewable matrix is free of flavoring agents (i.e., contains 0% by weight of the matrix).

The flavoring agents may comprise essential oils, synthetic flavors, or mixtures thereof including, but not limited to, oils derived from plants and fruits such as citrus oils, fruit essences, peppermint oil, spearmint oil, close oil, oil of wintergreen, anise and the like. Artificial flavoring components are also contemplated for use in the chewable matrix of the present disclosure. Those skilled in the art will recognize that natural and artificial flavoring agents may be combined in any sensory acceptable blend. All such flavors and flavor blends are contemplated for use in the chewable matrix of the present disclosure.

A chewable matrix of the present disclosure may also optionally have spray dried flavor as a partial or complete replacement of liquid flavor. This reduces the plasticizing or tackifying quality that liquid flavors provide. The amount of liquid flavor used would reduce to about 0.4% to about 2% by weight of the chewable matrix. The loading of the spray dried flavor in the chewable matrix can be approximately 20% active. The amount of spray dried flavor may be used up to about 2% by weight of the chewable matrix. Preferably, spray dried flavor is used in amounts ranging from about 0.2% to about 2% by weight of the chewable matrix. Even more preferably, spray dried flavor is used at about 1% by weight of the chewable matrix.

Optional ingredients such as colors, emulsifiers and pharmaceutical agents, coolants, oral sensates, active agents, antimicrobials, tooth whitening agents, medicaments, breath freshening agents, nutritional supplements, wellness agents, weight loss agents, and combinations thereof may be added to the chewable matrix. Colorants may typically include FD&C type lakes, plant extracts, fruit and vegetable extracts and titanium dioxide.

In one particular embodiment, the chewable matrix of the present disclosure comprises high quantities of low molecular weight PVAc and de-oiled lecithin. In one embodiment, the chewable matrix comprises low molecular weight PVAc in an amount of from about 5% to about 40% by weight of the matrix, or from about 10% to about 20% by weight of the matrix, and de-oiled lecithin in an amount of from about 13% to about 50%, from 20% to about 50%, or from about 30% to about 40% by weight of the matrix. In one embodiment, the chewable matrix is furthermore essentially free of filler. In one embodiment, the chewable matrix is free of filler. In another embodiment, the chewable matrix further comprises terpene resin. In one embodiment, the terpene resin is present in an amount of from about 10% to about 35% by weight of the matrix. In one embodiment, the chewable matrix comprises bulking agents in an amount of 60% or less by weight of the matrix. In one embodiment, the chewable matrix is free of flavoring agents.

In another embodiment, the chewable matrix comprises a low molecular weight PVAc and a low molecular weight elastomer. In one such embodiment, the PVAc has a molecular weight of 17,000 daltons or less, and the elastomer has a molecular weight of 100,000 daltons or less. In one such embodiment, the chewable matrix further is essentially free of high molecular weight elastomers (e.g., elastomers having a molecular weight of greater than 100,000 daltons). In another such embodiment, the chewable matrix is free of high molecular weight elastomers. In one embodiment, the chewable matrix comprises low molecular weight PVAc in an amount of from about 13% to about 35% by weight of the matrix, and low molecular weight elastomer (such as low molecular weight polyisobutylene and/or low molecular weight butyl rubber), in an amount of from about 3% to about 17% by weight of the matrix. In one embodiment, the chewable matrix comprises bulking agents in an amount of 60% or less by weight of the matrix. In one embodiment, the chewable matrix is free of flavoring agents.

The chewable matrix of the present disclosure is typically prepared by adding an amount of the elastomer (when included), PVAc, filler (when included) and elastomer solvent (when included) to a heated sigma blade mixer with a front to rear speed ratio of about 1:1 to about 2:1.

Compounding typically begins to be effective once the ingredients have become homogenous. Anywhere from 15 minutes to 90 minutes may be the length of compounding time. Preferably, the time of compounding is from 20 minutes to about 60 minutes. The amount of added plasticizer depends on the level of elastomer and other elastomer present. If too much elastomer plasticizer is added, the initial mass becomes over plasticized and not homogeneous.

Continuous processes using mixing extruders may also be used to prepare the chewable matrix. After the initial ingredients have massed homogeneously and been compounded for the time desired, the balances of the ingredients are added in a sequential manner until a completely homogeneous molten mass is attained. Typically, any remainder of elastomer and plasticizer are added after the initial compounding time. The optional waxes and any oils are typically added after the elastomer and plasticizers. Then the mass is allowed to become homogeneous before discharging.

The processes disclosed herein for manufacturing the chewable matrix are similar to manufacturing processes commonly used to formulate chewable polymer-based confectionery products. For example, U.S. Pat. No. 6,238,710, herein incorporated by reference, claims a method for continuous chewing gum base manufacturing. The method entails compounding all ingredients in a single extruder. U.S. Pat. No. 6,086,925 discloses the manufacture of chewing gum base by adding a hard elastomer, a filler and a lubricating agent to a continuous mixer. U.S. Pat. No. 5,419,919 discloses continuous gum base manufacture using a paddle mixer by selectively feeding different ingredients at different locations on the mixer. Yet another U.S. Pat. No. 5,397,580 discloses continuous gum base manufacture wherein two continuous mixers are arranged in series and the blend from the first continuous mixer is continuously added to the second continuous mixer. The processes disclosed in these patents may be utilized to form the chewable matrix of the present disclosure.

Typical base batch processing times may vary from about one to about three hours, preferably from about 1.5 to 2.5 hours, depending on the formulation. The final mass temperature when discharged may be between 50° C. and 130° C. and preferably between 70° C. and 120° C. The completed molten mass is emptied from the mixing kettle into coated or lined pans, extruded or cast into any desirable shape and allowed to cool and solidify. Those skilled in the art will recognize that many variations of the above described procedure may be followed.

In the alternative continuous process, ingredients are added continuously at various points along the length of the extruder. In this case, the transit time through the extruder would be substantially less than an hour.

In embodiments where the chewable matrix includes water-soluble ingredients, the water insoluble portion of the matrix may first be prepared as described above. The water-insoluble portion may then be melted and added to a running mixer. The mixer may be any commercially available mixer known in the confectionery arts. Color or emulsifiers may also be added at this time. A softener, such as glycerin, may also be added at this time, along with syrup and a portion of the bulking agent/sweetener. Further portions of the bulking agent/sweetener may then be added to the mixer. A flavoring agent is typically added with the final portion of the bulking agent/sweetener. A high-intensity sweetener is preferably added after the final portion of bulking agent and flavor have been added.

Although generally the water insoluble portion may be melted before adding to the mixture, in some embodiments, the water insoluble portion is not melted prior to adding it to the mixer.

According to certain non-limiting embodiments the entire mixing procedure typically takes from five to fifteen minutes. In another non-limiting embodiment of the method according to the present disclosure, longer mixing times may sometimes be required. Those skilled in the art will recognize that many variations of the above described procedure may be followed. Again, one specifically contemplated embodiment is the use of an extruding mixer for continuous processing. In such a process, ingredients are added continuously at various points along the length of the extruder while homogeneously mixed gum continuously issues from the discharge end of the extruder. U.S. Pat. No. 6,017,565, herein incorporated by reference, discloses a continuous manufacture process which automatically and continuously feeds ingredients into an apparatus, mixes, and discharges the desired end product. The end product is automatically dusted, rolled scored and wrapped. U.S. Pat. No. 5,543,160 discloses a manufacturing process using high efficiency continuous mixing which does not require separate manufacture of gum base.

After mixing, the chewable matrix is formed into a final product shape using well known techniques which may employ extrusion, rolling, sheeting, scoring or forming. The final product shape may be stick, tabs, chunks, pellets, balls or any other desired shape.

Pellet and ball forms, among others, may optionally be pan coated. Conventional panning procedures generally coat with sucrose, but recent advances in panning have allowed the use of other carbohydrate materials to be used in the place of sucrose. Some of these components include, but are not limited to, erythritol, sorbitol, dextrose, maltose, xylitol, hydrogenated isomaltulose and other new polyols or a combination thereof. These materials may be blended with panning modifiers including, but not limited to, gum arabic, maltodextrins, corn syrup, gelatin, cellulose type materials like carboxymethyl cellulose or hydroxymethyl cellulose, starch and modified starches, vegetable gums like alginates, locust bean gum, guar gum and gum tragacanth, insoluble carbonates like calcium carbonate or magnesium carbonate, and talc. Erythritol also acts as a panning modifier with other panning materials to improve product quality. Anti-tack agents may also be added as panning modifiers, which allow the use of a variety of carbohydrates and sugar alcohols to be used in the development of new panned or coated products. Flavors may also be added with the erythritol sweetener to yield unique product characteristics.

If the chewable matrix is in a pellet form and is to be coated, the initial coating syrup should have higher binder levels, e.g. gum Arabic or gum tallah, in the pre-coat, because conventional pre-coat does not stick to the pellet. The increase of a binder allows for the appropriate adherence of the pre-coat. The present chewable matrix may be coated in amount ranging from about 30% to about 38%. Preferably, the coating is present at about 32% to about 36%.

### Microorganisms

The present disclosure provides for methods of detecting, identifying, and/or quantitating microorganisms adhered to or entrapped within a chewable matrix in a subject. In addition, the disclosure provides for methods of determining the microorganism profile of a sample of saliva from a subject, or of the oral cavity of a subject. A profile of the microorganisms of a saliva sample may be used to determine susceptibility or risk of the organism for developing dental caries or periodontal disease, or other diseases, conditions, or infections, regardless if the microorganisms are currently causing an infection.

“Microorganisms” or “microbes” refer to microscopic organisms which may be single celled or multicellular organisms and may by pathogenic or commensal to the host organism. The disclosure provides for methods of detecting and quantitating microorganisms adhered to or entrapped within a chewable matrix of the present disclosure and these microorganisms include bacteria, viruses, fungi and protozoa. The term “type of microorganism” refers to a single family, genus, species, strain, serotype or serogroup of microorganism depending on identification method.

For example, the disclosure provides for methods of detecting, identifying and quantitating bacteria adhered to or entrapped within a chewable matrix. The term “type of bacteria” refers to a single family, genus, species, strain, serotype or serogroup of bacteria depending on identification method. The bacteria may be gram negative or gram positive bacteria, and anaerobic or aerobic bacteria. The bacteria may be a cocci (spherical) or bacilli (rod-shaped). The disclosure particularly contemplates methods of detecting oral bacteria such as commensal oral bacteria and pathogenic oral bacteria such as streptococci, lactobacilli, staphylococci, corynebacteria, actinomcyes sp., fusobacterium sp. and various anaerobes in particular bacteroides. Exemplary oral bacteria include Streptococcus mutans, Streptococcus oralis, Streptococcus salivarius, Actinomyces naeslundii, Streptococcus sanguinis, Porphyromomas gingivalis, Porphymomas intermedia, Bacteroides forsythus, Tanneraella. forsythia, Campylobacter rectus, Eubacerium nodatum, Peptostreptococcus micros, Streptococcus intermedius, Aggregetibacter actinomycetemcomitans, Treponema denticola, Eikenella corrodens, Capnocytophaga gingivalis, Streptococcus gordonii, Veillonella parvula, Fusobacterium nucleatum, Prevotella intermedia, Lactobacillus salivarius, Scardovia wiggsiae, Streptococcus salivarius and Streptococcus sobrinus.

The disclosure also provides for methods of detecting, identifying and quantitating viruses attached to the chewable matrix. The term “type of virus” refers to a single family, genus, species, strain, serotype or serogroup of virus depending on identification method.

The virus may be a member of the Herpesvirus family such as the Human Herpes Virus (HHV) including HHV-1 (also known as herpes simplex virus (HSV)-1), HHV-2 (HSV-2), HHV-3 (also known as varicella-zoster virus), HHV-4 (Epstein-Barr virus), HHV-5 (cytomegalovirus), HHV-6, HHV-7 and HHV-8.

The virus may be a member of the Picornaviridae family (Enterovirus genus) such as poliovirus, group A coxsackievirus, group B coxsackievirus, or echovirus. In particular, the virus may cause hand-foot-mouth disease such as coxsackievirus A16, A5, A7, A10, B2 and B5, a virus that causes herpangina such as coxsackievirus A1-6, A8, A10, and A22 and Enterovirus 71 (EV-71).

The virus may be a member of the Papovaviridae family such as the Human Papillomavirus family (HPV) including HPV-16, HPV-18, HPV-33 and HPV-35. The virus may be a member of the Paramyoxvirus family (Rubularivurs genus) such as mumps virus, Newcastle disease virus, human parainfluenza virus type 2, 4a and 4b. The virus may be a member of the Paramyxovirus family (Morbillivirus genus). In addition, the virus may be a member of the Togavirus family (Rubivirus genus). The virus may also be canine oral Papilloma virus, feline calicivirus, and feline herpesvirus.

The virus may also be an influenza virus such as human influenza virus A such as H1N1 and H3N3, human influenza virus B and human influenza virus C. The virus can be a virus that causes the common cold such as rhinovirus.

The virus may also be a coronavirus, such as severe acute respiratory syndrome coronavirus (SARS-CoV) or severe acute respiratory syndrome coronavirus 2 (SARS-CoV-2), which causes COVID-19 respiratory disease.

Certain herpes viruses (herpes simplex and varicella-zoster virus, the cause of chickenpox and shingles) are known causes of gingivitis. Other herpes viruses (cytomegalovirus and Epstein-Barr) may also play a role in the onset or progression of some types of periodontal disease, including aggressive and severe chronic periodontal disease. All herpes viruses go through an active phase followed by a latent phase and possibly reactivation. These viruses may cause periodontal disease in different ways, including release of tissue-destructive cytokines, overgrowth of periodontal bacteria, suppressing immune factors, and initiation of other disease processes that lead to cell death.

The methods of the disclosure may detect a fungus such as Candida e.g. Candida albicans, Aspergillus, Cryptococcus neoformans, Cryptococcus gattii, Histoplasma capsulatum, Blastomyces dermatitidis, Paracoccidioides brasiliensis, Coccidiodes immites and Zygomycota.

The methods of the disclosure may detect protozoa such as Entamoeba gingivalis and Trichomonas tenax.

The disclosure also contemplates detecting microorganisms that cause or are related to the oral diseases and disorders such as periodontal disease (inflammation or infection of gum tissue) including, but not limited to, chronic periodontitis and acute adult periodontitis, gingivitis (acute or chronic), Vincent angina, dental caries, herpesvirus infection, primary herpetic gingivostomatitis, or oral herpes (cold sores and canker sores), genital herpes, varicella-zoster virus infection e.g. chicken pox or shingles, influenza, COVID-19, common cold, venereal disease, mononucleosis, coxsackievirus infection such as hand-foot-mouth disease, herpangina, acute lymphonodular pharyngitis, mumps, measles (reubeola), rubella (German measles), African Burkitt lymphoma, nasopharyngeal carcinoma, oral hairy leukoplakia, roseola infantum, Karposi sarcoma, Candidiasis, acute pseudomemranous candidosis (thrush), acute atrophic (erythematous) candidosis, chronic hyperplastic candidosis, and chronic atrophic (erythematous) candidosis, aspergillosis, cryptococcosis, Histoplasmosis (also known as Cave disease, Darling's disease, Ohio valley disease, Reticuloendotheliosis, Spelunker's Lung and Caver's disease, Blastomycosis (also known as North American blastomycosis, Blastomycetic dermatitis, or Gilchrist's disease) Paracoccidioidomycosis (also known as Brazilian blastomycosis, South American blastomycosis, Lutz-Splendore-de Almeida disease and Paracoccidioidal granuloma), mucormycosis (after Mucorales), Scardovia wiggsiae infection; phycomycosis (after Phycomycetes) and basidiobolomycosis (after Basidiobolus) and zygomycosis (mucormycosis). The present disclosure also provides for methods of diagnosing any condition, disease or disorder that are caused by or related to the presence of a microorganism adhered to or entrapped within the chewable matrix of the present disclosure.

### Mammalian Cells

In some embodiments, methods of the disclosure may also be used to detect nucleic acids from a mammalian cell, such as human cells, canine cells, feline cells, murine cells, rat cells, bovine cells, equine cells, sheep cells, goat cells, primate cells, cells from aquatic mammals such as whales and dolphins and cells from other exotic mammals, that are adhered to or entrapped within a polymer. The mammalian cells that may be detected include epithelial cells, squamous cells, fibroblasts, keratinocytes, odontoblasts, ameloblasts, cells within a taste bud such as sensory cells, support (or sustentacular) cells, stem cells, basal cells, cells within the salivary gland such as serous cells and mucous cells.

Furthermore, the cells may be cancer cells such as from a tumor that has developed on the surface of the tongue, mouth, lips, pharynx, tonsils or gums such as oral squamous cell carcinoma, salivary gland adenocarcinoma, mucoepidermoid carcinoma, adenoid cystic carcinoma, sarcomas (tumors arising from bone, cartilage, fat, fibrous tissue or muscle) lymphoma or melanoma. The cells may be precancerous cells from subjects suffering from precancerous conditions such as leukoplakia or erythroplakia.

### Kits

In another aspect, the disclosure provides for kits for carrying out any of the preceding methods described herein. In particular, the disclosure provides for kits comprising a chewable matrix of the present disclosure and/or components for saliva sample collection and/or detection and/or quantification of microorganisms adhered to or entrapped within masticated chewable matrix according to any of the methods disclosed herein.

In one embodiment, the disclosure provides for kits comprising components for detection and/or quantification of microorganisms and/or nucleic acids from a cell or microorganism in a saliva sample and/or that are adhered to or entrapped within a chewable matrix according to any of the methods of the disclosure. The kits may comprise oligonucleotide primers for amplifying a fragment of the nucleic acid specific for a microorganism of interest, a mammalian cell, such as human cells, or a cancer cell. The kits may further comprise universal forward and/or reverse primers for amplifying the nucleic acid. The kits may also comprise instructions for carrying out the methods of the disclosure.

The kits of the disclosure may also comprise the components necessary to carry out PCR or other amplification methods. For example, the kit may contain one or more of the following: Taq polymerase or another thermostable polymerase, ATP, suitable amounts of each of the four deoxyribonucleoside triphosphates (dNTPs), and buffers, salts such as MgCl₂, preservatives, reducing agents or water.

Kits may also comprise any the components necessary to carry out the detection assays such as the organic solvent and a buffered solution. For example, the kits may optionally comprise an organic solvent such as chloroform, xylene or toluene, and/or a buffered solution that is conventional in the field of molecular biology and in which the nucleic acids are stable, such as Tris buffer, TBE buffer (Tris-borate-EDTA) and TAE buffer (Tris-acetate-EDTA). In a particular embodiment, the buffered solution is Tris buffer that comprises tris(hydroxymethyl)aminomethane. Tris buffer is also known as Tris base, Trizma, Trisamine, THAM, Tromethamine, Trometamol and Tromethane. In addition, the kits of the disclosure may optionally further comprise buffers for PCR amplification, dNTP's and buffers for gel loading, such as chloroform and/or Tris buffer. The kits may comprise buffers, loading dyes, gels, molecular weight markers, membranes, filters, blocking buffers and detection reagents for Northern Blot analysis, Southern Blot analysis, slot-blot analysis or in situ hybridization analysis and any other methods so long as they may identify the source or the presence/expression of a particular gene sequence.

Kits may further comprise one or more test tube or other container which may be used to store a free saliva sample collected following mastication of the chewable matrix (i.e., a saliva sample collected via spitting) and/or to store the masticated chewable matrix prior to further analysis. In one embodiment, the kit may comprise a single container for storage of a free saliva sample collected following mastication of the chewable matrix, for storage of the masticated chewable matrix, or for storage of both the free saliva sample collected following mastication of the chewable matrix and the masticated chewable matrix. In another embodiment, the kit may comprise at least 2 containers, wherein one container is for storage of the masticated chewable matrix and one container is for storage of a free saliva sample collected following mastication of the chewable matrix.

Other aspects and advantages of the present disclosure will be understood upon consideration of the following illustrative examples.

### EXAMPLES

### Example 1

Examples of a chewable matrix of the present disclosure (Matrix B and Matrix C) were prepared, and the ability of the matrix to absorb saliva following 2, 3, 5, or 10 minutes of mastication was determined. The results were compared to those achieved with a conventional polymer-based confectionery product (Matrix A, Control). The formulations for the control, Matrix B, and Matrix C are set forth below in Table 2.

TABLE 2

| | Matrix A | Matrix B | Matrix C |
|---|---|---|---|
| | (Control) | (Experimental) | (Experimental) |
| Calcium Carbonate | 6.1124 | 0.1848 | 6.55 |
| Butyl Rubber | 2.3632 | 4.9104 | 2.500 |
| Polyisobutylene | 2.8756 | | |
| Terpene Resin | | 13.3452 | |
| Rosin Ester | 4.2784 | | 4.549 |
| Polyvinyl acetate¹ | 4.6564 | 15.6464 | 5.00 |
| Hydrogenated Vegetable | 5.0288 | 4.3824 | 5.400 |
| Oil | | | |
| Vegetable Oil | 1.7752 | 2.9436 | 2.00 |
| Mono and Diglycerides | 0.9016 | 2.5652 | 1.00 |
| Soy Lecithin | 0.1000 | | |
| De-oiled Soy Lecithin | | 40.0000 | 7.00 |
| Sorbitol | 61.3500 | | 37.000 |
| Sorbitol Syrup | | | 26.000 |
| Maltitol | | 11.0000 | |
| Glycerin | 8.7500 | 5.0000 | |
| Peppermint Flavor | 1.8000 | | |
| BHT | 0.0084 | 0.0220 | 0.001 |
| | 100.0000 | 100.0000 | 100.000 |

¹low molecular weight PVAc

Matrix A, Matrix B, and Matrix C were separately masticated by a subject diagnosed with infection with COVID-19 respiratory disease (n=1), which is caused by infection with SARS-CoV-2. The weight of the matrix was measured prior to mastication, and following mastication for 2, 3, 5, or 10 minutes, and the moisture content retained in the cud following mastication was calculated. The results are set forth below in Table 3.

TABLE 3

| | | | | Difference | decrease/ | Moisture % | Moisture % |
|---|---|---|---|---|---|---|---|
| | Chew | Starting | End | in start/end | increase in | (based on | (based on |
| Formulation | time (min) | weight (g) | weight (g) | weight (g) | weight (%) | start weight) | cud weight) |
| Control | 3 | 2.0525 | 0.9085 | −1.1440 | −55.7369 | 5.66 | 12.81 |
| (Matrix A) | 5 | 2.0383 | 0.7938 | −1.2445 | −61.0558 | 5.65 | 14.54 |
| | 10 | 1.9979 | 0.7321 | −1.2658 | −63.3565 | 5.43 | 14.86 |
| Matrix B | 3 | 2.0523 | 2.5216 | 0.4693 | 22.8670 | 27.14 | 22.09 |
| | 5 | 2.0591 | 2.5742 | 0.5151 | 25.0158 | 35.94 | 23.44 |
| | 10 | 1.9951 | 3.1206 | 1.1255 | 56.4132 | 60.36 | 38.59 |
| Matrix C | 2 | 1.8153 | 1.3201 | −0.4952 | −27.2800 | N/A | 15.23 |

As can be seen from these results, Matrix B and Matrix C had a higher moisture content following mastication than did control (Matrix A). This indicates that Matrix B and Matrix C collected a greater amount of saliva following mastication than did the control.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A chewable matrix comprising low molecular weight polyvinyl acetate in an amount of from about 5% to about 40% by weight of the matrix, and de-oiled lecithin in an amount of from about 13% to about 50% by weight of the matrix.

2. The chewable matrix of claim 1, wherein the matrix comprises low molecular weight polyvinyl acetate in an amount of from about 10% to about 20% by weight of the matrix.

3. The chewable matrix of claim 1, wherein the matrix comprises de-oiled lecithin in an amount of from about 30% to about 40% by weight of the matrix.

4. The chewable matrix of claim 1, further comprising terpene resin.

5. The chewable matrix of claim 4, wherein the matrix comprises terpene resin in an amount of from about 10% to about 35% by weight of the matrix.

6. The chewable matrix of claim 1 wherein the matrix is essentially free of elastomers.

7. A chewable matrix comprising low molecular weight polyvinyl acetate and a low molecular weight elastomer.

8. The chewable matrix of claim 7, wherein the low molecular weight polyvinyl acetate has a molecular weight of 17,000 daltons or less.

9. The chewable matrix of claim 7, wherein the low molecular weight elastomer is selected from the group consisting of low molecular weight polyisobutylene, low molecular weight butyl rubber, and combinations thereof.

10. The chewable matrix of claim 7, wherein the chewable matrix is essentially free of high molecular weight elastomers.

11. The chewable matrix of claim 10, wherein the chewable matrix is free of high molecular weight elastomers.

12. The chewable matrix of claim 7, wherein the chewable matrix comprises low molecular weight PVAc in an amount of from about 13% to about 35% by weight of the matrix.

13. The chewable matrix of claim 7, wherein the chewable matrix comprises low molecular weight elastomer in an amount of from about 3% to about 17% by weight of the matrix.

14. A chewable matrix comprising low molecular weight polyvinyl acetate and at least one plasticizer, wherein the matrix is essentially free of elastomers.

15. The chewable matrix of claim 14, wherein the matrix comprises low molecular weight polyvinyl acetate in an amount of from about 5% to about 95% by weight of the matrix.

16. The chewable matrix of claim 15, wherein the matrix comprises the low molecular weight polyvinyl acetate in an amount of from about 40% to about 99% by weight of the matrix.

17. The chewable matrix of claim 14, wherein the matrix comprises plasticizer in an amount of from about 0.5% to about 15% by weight of the matrix.

18. The chewable matrix of claim 14, wherein the plasticizer is selected from the group consisting of water, triacetin, an acetylated monoglyceride, and combinations thereof.

19. The chewable matrix of claim 1, wherein the matrix comprises bulking agents in an amount of 60% or less, by weight of the matrix.

20. The chewable matrix of claim 1, wherein the matrix is free of flavoring agents.

21. The chewable matrix of claim 1, wherein the chewable matrix is essentially free of filler.

22. A method of collecting a saliva sample from a subject, the method comprising contacting the oral cavity of the subject with the chewable matrix of claim 1.

23. A method of detecting an illness or infection in a subject, the method comprising contacting the oral cavity of the subject with the chewable matrix of claim 1.

24. The method of claim 23 further comprising extracting microorganisms from the chewable matrix by sonicating the chewable matrix under conditions that remove the microorganisms from the chewable matrix and deposit the microorganisms in a suspension.

25. The method of claim 24, wherein at least a portion of the suspension is contacted with a solid support under conditions that promote colony formation.

26. The method of claim 23 further comprising extracting microorganisms from the chewable matrix by contacting the chewable matrix with a solution comprising at least one selected from the group consisting of an organic solvent, a buffer, and water.

27. The method of claim 23 further comprising extracting nucleic acids from the chewable matrix by contacting the chewable matrix with a solution comprising at least one selected from the group consisting of an organic solvent, a buffer, and water, and optionally subjecting to a mechanical mixer to break up the chewable matrix.

28. The method of claim 27, wherein the organic solvent is chloroform.

29. The method of claim 27, further comprising the step of identifying the source of the nucleic acids extracted from the chewable matrix using polymerase chain reaction.

30. The method of claim 29, wherein the identified source of the nucleic acids is a virus.

31. The method of claim 30, wherein the virus is severe acute respiratory syndrome coronavirus 2 (SARS-CoV-2).

32. The method of claim 27, further comprising the step of quantifying the nucleic acids extracted from the chewable matrix using quantitative polymerase chain reaction.

33. The method of claim 23, further comprising detecting microorganisms adhered to or entrapped within the chewable matrix following mastication by (a) dissolving at least a portion of the chewable matrix in a solution comprising at least one selected from the group consisting of an organic solvent, a buffer, and water, (b) extracting nucleic acids from the microorganisms, and (c) amplifying the nucleic acids using at least one oligonucleotide primer specific for the microorganism.

34. The method of claim 34, wherein the nucleic acids are amplified using polymerase chain reaction.

35. The method of claim 23, further comprising quantitating microorganisms adhered to or entrapped within the chewable matrix by (a) sonicating the chewable matrix under conditions that remove the microorganisms from the chewable matrix and deposit the microorganisms in a suspension, (b) contacting at least a portion of the suspension with a solid-support under conditions that promote colony formation, and (c) quantitating the colonies formed on the solid-support, wherein the number of colonies indicates the total quantity of microorganisms.

36. The method of claim 36, wherein the microorganisms are selected from the group consisting of bacteria, virus, fungus, protozoa, and combinations thereof.

37. A kit for detection of illness or infection, the kit comprising
the chewable matrix of claim 1;
at least one container for collection of a saliva sample following mastication of the chewable matrix; and
components for detection and/or quantification of microorganisms and/or nucleic acids.

38. The kit of claim 38, further comprising at least two containers for collection of a saliva sample following mastication of the chewable matrix.
